(19) United States
(12) Patent Application Publication
(10) Pub. No.: US 2024/0180828 A1
(43) Pub. Date: Jun. 6, 2024

(54) COMPOSITIONS

(71) Applicant: VITUX GROUP AS, Oslo (NO)

(72) Inventors: Morten J DILLE, Oslo (NO); Tuna BAYDIN, Oslo (NO); Magnus HATTREM, Oslo (NO); Kurt Ingar DRAGET, Oslo (NO)

(21) Appl. No.: 18/286,433

(22) PCT Filed: Apr. 14, 2022
(86) PCT No.: PCT/GB22/50962

§ 371 Date: Oct. 11, 2023

(30) Foreign Application Priority Data
Apr. 16, 2021 (GB) 2105461.4

(51) Int. Cl. A61K 9/107; A61K 9/00; A61K 47/36; A61K 47/42; A61K 47/44

(52) U.S. Cl. CPC A61K 9/107; A61K 9/0053; A61K 47/36; A61K 47/42; A61K 47/44

(57) ABSTRACT

The invention relates to compositions for oral administration which are provided in the form of gelled oil-in-water emulsions, to methods for their preparation and to their use as pharmaceuticals and nutraceuticals. In particular, it relates to orally administrable. gelled oil-in-water emulsions which are self-supporting. viscoelastic solids having a gelled aqueous phase comprising a gelling agent which is agar, and wherein said emulsion is stabilised by a surfactant which is a plant-based protein. plant-based polysaccharide or derivative thereof.

### FIELD OF THE INVENTION

The present invention generally relates to compositions for oral administration which are provided in the form of gelled oil-in-water emulsions, to methods for their preparation and to their use as pharmaceuticals and nutraceuticals. The compositions are soft, yet chewable, and can be provided in a unit dosage form which is easy to swallow. More specifically, the invention relates to oral compositions which are acceptable to patients and consumers that wish to abstain from the consumption of animal by-products, for example those that follow a vegetarian diet or who are vegan. It also relates to oral compositions that are acceptable to pescetarians.

### BACKGROUND OF THE INVENTION

Soft chewable dosage forms are an alternative to traditional oral administration forms such as tablets, capsules, elixirs and suspensions. They are easier to swallow than tablets and capsules and are particularly suitable for the pediatric and elderly population as well as those that suffer from dysphagia. Such dosage forms are a popular choice for dietary supplements which contain vitamins and/or minerals (so-called “nutraceuticals”), and are also suitable for the delivery of active pharmaceutical ingredients (APIs). Active components (nutraceutical or pharmaceutical) may be present in the form of lipids in gelled oil-in-water emulsions, as dispersed particulates or dissolved in the oil or aqueous phase of such emulsions.

A range of gelling agents can be used in the preparation of soft chewable dosage forms, such as gelled oil-in-water emulsions, however gelatin is by far the most widely used due to its availability, ease of use and its sol-gel transition temperature. Gelatin is produced by partial hydrolysis of collagen found in the skin, bones and connective tissue of animals and is most commonly derived from pork, bovine and fish sources. The sol-gel transition temperature of a gelatin generally corresponds to the body temperature of the animal from which it is obtained. Gelatins from mammalian sources therefore have transition temperatures which are similar to human body temperature, resulting in gels which are solid at room temperature but which melt in the mouth once ingested. Gelatin-based dosage forms thus provide a pleasant ‘melt-in-the mouth’ texture or “mouthfeel”. Gelatin also provides fast and consistent dissolution kinetics of a dosage unit in the gastrointestinal tract which can be beneficial to promote rapid uptake of any active components.

Gelatin has significant surface active properties which allows it to act as an emulsifier as well as a gelling agent. This makes it a particularly good choice for use as a gelling agent to produce oil-in-water emulsions which are chewable. Gelatin-based emulsions typically experience an “active filler effect” in which the droplets of oil interact strongly with the surrounding gel network and are generally referred to as “active fillers”. When the oil droplets are sufficiently small, this interaction between the gel network and the oil droplets increases the storage modulus of the gelled emulsion compared to an oil-free gel, i.e. the gel alone. In contrast, oil droplets which are distributed throughout a gel with little or no interaction with the gel network are known as “inactive fillers” and result in a modulus for the gelled emulsion which is lower than that of the gel alone. When oil droplets of a gelled emulsion are present as “inactive fillers”, the emulsion may not be stable over time. That can lead to destabilization of the emulsion and ‘sweating’ of oil.

Despite the many advantageous properties of gelatin for use in the production of soft chewable dosage forms, its animal origin makes it unacceptable to many patients and consumers due to their religious beliefs or dietary choices. As an animal by-product, gelatin is not acceptable to vegans for example.

Gelling agents that are not of animal origin and which have previously been proposed for use in the production of soft chewable dosage forms, such as gelled oil-in-water emulsions, include non-proteinaceous materials such as alginates, carrageenans and pectins. However, the gelling properties of these materials can be difficult to control due to the need for their complexation with metal ions, temperature change and/or pH adjustment to produce the desired ‘gel’. This is not ideal in the context of a dosage form which is to be manufactured on a commercial scale.

An alternative gelling agent which is widely used in food and other non-food applications is agar. Agar is extracted from marine red algae and comprises a polysaccharide containing galactose sub-units. It is a thermosetting polymer which gels at about 30-45° C. Agar melts at about 85-90° C. and once melted it retains a liquid state until cooled to 40° C. Due to its large hysteresis between gelling and melting temperatures it has the potential for use in the large scale production of dosage units formed from gelled oil-in-water emulsions. However, unlike gelatin which produces soft, flexible gels that can withstand a high degree of compression before they break, agar-based gels are hard and brittle. Whereas a gelatin-based gel might withstand up to 70-90% compression before it breaks, for example, an agar-based gel will typically fragment under a deformation of as little as 20%. This severely restricts its use in the production of any dosage unit that needs to be soft and chewable and have a pleasant mouthfeel.

Unlike gels based on gelatin, agar-based gels are also prone to syneresis, i.e. spontaneous release of water from the gel on ageing. Gels are a 3D network of polymers which cross-link with one another trapping water within their structure. If the polymer network is not disturbed, the water remains in place. Over time, however, the polymers which form the gel may contract or alter their conformation causing water to be expelled and shrinkage of the gel. Oozing of water out of the gel is known as “syneresis” and this must be minimised in any oral dosage unit due to the need for it to have an adequate shelf-life, i.e. it should remain stable over an extended period of time. One of the ways in which the problem of syneresis of agar gels has traditionally been addressed is by increasing the agar concentration. However, that can lead to a harder, more solid and more brittle gel which is undesirable when seeking to provide a soft, chewable dosage form.

There is thus a continuing need for alternative soft, yet chewable, compositions for the oral delivery of pharmaceuticals and/or nutraceuticals that are suitable for vegetarians, pescetarians and vegans. In particular, there is a need for such compositions that can provide an acceptable alternative to conventional gelatin-based oil-in-water emulsions in terms of their “chew” and mouthfeel characteristics. Such compositions should be capable of manufacture on a commercial scale and have adequate stability (i.e. shelf-life) for use as pharmaceutical and/or nutraceutical products.

The present invention addresses at least some of these needs.

### SUMMARY OF THE INVENTION

The Applicant now proposes gelled oil-in-water emulsions that are acceptable to patients and consumers that are vegetarian, pescetarian or vegan, in particular to those that follow a vegetarian or vegan diet. The emulsions employ agar as a gelling agent and are stabilised using certain plant-based surfactants. Specifically, the emulsions are stabilised by at least one surface-active protein or polysaccharide derived from a plant or a derivative thereof. When used to stabilise agar-based, gelled oil-in-water emulsions the Applicant has found that these high molecular weight (i.e. “macromolecular”), plant-based surfactants are advantageous compared to low molecular weight surfactants. In particular, they have found that these macromolecular surfactants provide gelled emulsions that are stable and which possess desirable rheology characteristics for the oral delivery of active agents in a soft, yet chewable, dosage form.

In one aspect the invention provides an orally administrable, gelled oil-in-water emulsion which is a self-supporting, viscoelastic solid having a gelled aqueous phase comprising a gelling agent which is agar, and wherein said emulsion is stabilised by a surfactant which is a plant-based protein, a plant-based polysaccharide, or a derivative thereof.

In another aspect the invention provides a method for the preparation of a gelled oil-in-water emulsion as herein described, said method comprising the steps of: forming an oil phase which comprises one or more physiologically tolerable lipids; forming an aqueous phase comprising a gelling agent which is agar; combining said oil phase and said aqueous phase to form an oil-in-water emulsion in the presence of a surfactant which is a plant-based protein, plant-based polysaccharide, or a derivative thereof; and allowing said emulsion to gel.

In a further aspect the invention provides a gelled oil-in-water emulsion as herein described for oral use as a medicament or for oral use in therapy.

In another aspect the invention provides a gelled oil-in-water emulsion as herein described which contains at least one pharmaceutically active component for oral use in the treatment of a condition responsive to said pharmaceutically active component.

In another aspect the invention provides the use of a pharmaceutically active component in the manufacture of a medicament for oral use in the treatment of a condition responsive to said pharmaceutically active component, wherein said medicament is provided in the form of a gelled oil-in-water emulsion as herein described.

In another aspect the invention provides a method of treatment of a human or non-human animal subject (e.g. a patient) to combat a condition responsive to a pharmaceutically active agent, said method comprising the step of orally administering to said subject a pharmaceutically effective amount of said agent in the form of a gelled oil-in-water emulsion as herein described.

In another aspect the invention provides the use of a gelled oil-in-water emulsion as herein described as a nutraceutical.

### DETAILED DESCRIPTION OF THE INVENTION

### Definitions

The term “gel” refers to a form of matter that is intermediate between a solid and a liquid. The formation of a “gel” will typically involve the association or cross-linking of polymer chains to form a three-dimensional network that traps or immobilises solvent (e.g. water) within it to form a sufficiently rigid structure that is resistant to flow at ambient temperature, i.e. at a temperature below about 25° C., preferably below about 20° C. In rheological terms, a “gel” may be defined according to its storage modulus (or “elastic modulus”), G′, which represents the elastic nature (energy storage) of a material, and its loss modulus (or “viscous modulus”), G″, which represents the viscous nature (energy loss) of a material. Their ratio, tan δ (equal to G″/G′), also referred to as the “loss tangent”, provides a measure of how much the stress and strain are out of phase with one another.

A material which is “viscoelastic” is characterised by rheological properties which resemble, in part, the rheological behaviour of a viscous fluid and, also in part, that of an elastic solid.

The gelled oil-in-water emulsions according to the invention are “self-supporting, viscoelastic solids”. This is intended to mean that they exhibit characteristics intermediate between those of a solid and a liquid, but have a dominant solid behaviour, i.e. they have rheological characteristics more similar to that of a solid than a liquid. A “solid dominant behaviour” cannot be diluted away (i.e. destroyed) by adding more solvent. In contrast, in the case of a weak (or entangled) gel lacking stable (i.e. long lived) intermolecular crosslinks, the entangled network structure of the gel can be removed by adding more solvent and can readily be destroyed even at very low shear rate/shear stress.

The gelled oil-in-water emulsions of the invention exhibit mechanical rigidity, yet in contrast to a solid they are deformable. Specifically, the gelled emulsions herein described have a storage modulus, G′, which is greater than their loss modulus, G″, (i.e. G′>G″) over a wide frequency range, for example in the frequency range from 0.001 to 10 Hz when measured at ambient temperature (i.e. at a temperature in the range of 18° C. to 25° C., e.g. at 20° C.) and 0.1% strain. Storage modulus and loss modulus may be measured using known methods, for example using a Kinexus Ultra+ Rheometer applying a C 4/40 measuring geometry. Storage modulus and loss modulus values are not expected to differ when measured using other types of rheometer within the linear viscoelastic range.

More specifically, the gelled oil-in-water emulsions herein described will have the following properties: G′>G″ over a frequency range of 0.001 to 10 Hz at 0.1% strain; and a storage modulus (G′) at ambient temperature (i.e. at a temperature in the range of 18° C. to 25° C., e.g. at 20° C.) in the range from 10 to 200,000 Pa, preferably 100 to 100,000 Pa, more preferably 500 to 50,000 Pa.

Weak gels will typically have a loss tangent, tan δ>0.1. For strong gels, or fully developed gels, G′»G″ and lower tan δ values (<0.1) are observed. The gelled oil-in-water emulsions herein described would generally be considered “strong gels” at ambient temperature, i.e. at a temperature in the range of 18° C. to 25° C., e.g. at 20° C.

As used herein, the term “gelled” refers to the formation of a “gel”. The term is used herein both in relation to the physical nature of the aqueous phase of the emulsion and that of the oil-in-water emulsion. As will be understood, the oil droplets act more or less like a solid when dispersed throughout the gelled aqueous phase of the oil-in-water emulsions which are the subject of the invention. The “gelled” nature of the aqueous phase is thus also a characteristic of the oil-in-water emulsion, i.e. it can also be considered “gelled” as described herein.

Unless otherwise defined, the term “liquid” as used herein refers to a substance which flows freely and which maintains a constant volume. It includes thickened liquids and viscous liquids which flow. A “liquid” has a loss modulus (G″) which is greater than its storage modulus (G′) and a loss tangent (tan δ) which is greater than 1.

As used herein, the term “surfactant” refers to a surface active compound or composition which is capable of reducing the interfacial tension between two immiscible liquids, e.g. at the interface between oil and water. Typically, a surfactant will be amphiphilic in nature and will comprise both hydrophobic and hydrophilic components. It may consist of a single component or may be a mixture of components. Where the surfactant is a mixture of components, the individual components will typically, though not necessarily, be similar in structure. To the extent that the surfactant for use in the invention is obtained from a natural product (i.e. from a plant or plant part), it will be understood that it will typically comprise a mixture of different components. The surfactant may be a naturally-occurring product obtained from a plant or part of a plant, or it may be a derivative thereof as described herein (i.e. it may be semi-synthetic).

As used herein, the term “fatty acid” refers to an un-branched or branched, preferably un-branched, hydrocarbon chain having a carboxylic acid (—COOH) group at one end, conventionally denoted the α(alpha) end. The hydrocarbon chain may be saturated or (mono- or poly-) unsaturated. By convention, the numbering of the carbon atoms starts from the α-end such that the carbon atom of the carboxylic acid group is carbon atom number 1. The other end, which is usually a methyl (—CH₃) group, is conventionally denoted ω (omega) such that the terminal carbon atom is the ω-carbon. Any double bonds present may be cis- or trans- in configuration. The nomenclature “ω-x” is used to signify that a double bond is located on the xth carbon-carbon bond, counting from the terminal carbon (i.e. the ω-carbon) towards the carbonyl carbon.

By “physiologically tolerable” is meant any component which is suitable for administration to a human or non-human animal body, in particular which is suitable for oral administration.

By “pharmaceutical” is meant any product intended for a medical purpose, e.g. for treating or preventing any disease, condition or disorder of a human or non-human animal body, or for preventing its recurrence, or for reducing or eliminating the symptoms of any such disease, condition or disorder. The use and production of a product as a “pharmaceutical” will be closely regulated by a government agency. It may, but need not, be prescribed by a physician. For example, it may be available “over the counter”, i.e. without a prescription.

“Treatment” or “treating” includes any therapeutic application that can benefit a human or non-human animal (e.g. a non-human mammal). Both human and veterinary treatments are within the scope of the present invention, although primarily the invention is aimed at the treatment of humans. Veterinary treatment includes the treatment of livestock and domestic animals (e.g. pets such as cats, dogs, rabbits, etc.). Treatment may be in respect of an existing disorder or it may be prophylactic.

In contrast to a pharmaceutical, a “nutraceutical” need not be the subject of regulatory approval. The term “nutraceutical” is used herein to refer to a product which is generally considered beneficial to maintain or augment the health and/or general well-being of a human or non-human animal subject. Such substances include, in particular, dietary supplements such as vitamins and minerals which are intended to augment the health of a subject (e.g. a human subject).

As will be understood, some substances may be considered both a “pharmaceutical” and a “nutraceutical”. Categorization of a substance as one or the other, or indeed both, may vary in different countries depending on local regulations relating to medicinal products. It may also be dependent on the recommended daily dosage of any given substance. Higher daily doses of certain vitamins such as vitamin D, for example, may be regulated as a pharmaceutical whereas lower daily dosages may be considered nutraceutical.

By “a pharmaceutical composition” is meant a composition in any form suitable to be used for a pharmaceutical purpose.

By a “nutraceutical composition” is meant a composition in any form suitable to be used for a nutraceutical purpose.

A “pharmaceutically effective amount” relates to an amount that will lead to the desired pharmacological and/or therapeutic effect, i.e. an amount of the agent which is effective to achieve its intended pharmaceutical purpose. While individual patient needs may vary, determination of optimal ranges for effective amounts of any active agent is within the capability of those skilled in the art.

A “nutraceutically effective amount” relates to an amount that will lead to the desired nutraceutical effect, i.e. an amount of the agent which is effective to achieve its intended nutraceutical purpose. While the individual needs of a subject may vary, determination of optimal ranges for effective amounts of any active agent is within the capability of those skilled in the art.

The term “capsule” is used herein to refer to a unitary dosage form having a casing or coating (herein referred to as the “capsule shell”) which encloses a gelled oil-in-water emulsion as herein defined.

As used herein, “water activity” is the partial vapour pressure of water in a composition at a specified temperature divided by the standard state partial vapour pressure of water at the same temperature. Water activity thus acts as a measure of the amount of free (i.e. unbound) water in a composition. Water activity may be measured by methods known to those skilled in the art, for example by using a Rotronic Hygrolab instrument.

As used herein, an “animal by-product” is intended to refer to any product derived from, isolated from, or purified from one or more parts of an animal body (e.g. bone, skin, tissue, meat, cartilage, hoof, horn, etc.). It is also intended to refer to any composition preparing by processing an animal by-product, for example, derivatised, functionalised, or otherwise chemically or physically modified, animal by-products. As used herein, an “animal by-product” is not intended to include milk, eggs, or any compound or composition that is derived from, isolated from, or purified from animal milk or animal eggs. The term “animal by-product” does not include any synthetic material, or any material obtained from any plant, fungal, bacterial or algal source.

As used herein, the term “vegetarian diet” generally refers to a diet that lacks any meat and which also lacks any animal by-product as herein defined. A “vegetarian diet” may include animal milk and animal eggs and any products derived, isolated or purified therefrom. Such a diet may also be generally known as an “ovo-lactovegetarian” diet or “lacto-ovovegetarian” diet which, in addition to food from plants, includes milk, cheese, other dairy products and eggs. A “pescetarian diet” refers to a diet in which the only source of meat is fish and seafood. A “vegan diet” refers to a diet that is totally vegetarian and which includes only food from plants (e.g. fruit, vegetables, grains, legumes, seeds and nuts). Any reference herein to a product, substance, composition or formulation which is “suitable for” a given diet means that it would be acceptable for those that follow that particular diet. The terms “vegetarian”, “pescetarian” and “vegan” are intended to refer to those who follow a vegetarian, pescetarian or vegan diet, respectively.

In a first aspect the invention provides an orally administrable, gelled oil-in-water emulsion which is a self-supporting, viscoelastic solid having a gelled aqueous phase comprising a gelling agent which is agar, and wherein said emulsion is stabilised by a surfactant which is a plant-based protein, a plant-based polysaccharide, or a derivative thereof.

The aqueous phase of the emulsion according to the invention comprises water and is gelled using agar as a gelling agent. The aqueous phase is also referred to herein as the “continuous phase” of the emulsion. The gelling agent may be a single type of gelling agent or it may be a mixture of different types of gelling agents. Where more than one gelling agent is used, at least one of the agents will be agar.

Agar is well known and used in the art, for example in food and other non-food applications. It is envisaged that any known type of agar may be used in the invention. As used herein, the term “agar” is intended to broadly define any product which contains a hydrocolloidal polysaccharide extracted from red seaweed, i.e. a seaweed of the family Rhodophyceae. The hydrocolloidal polysaccharide present in agar contains one or more polymers made up of subunits of galactose. Sources of agar include seaweeds belonging to the following genera: Gelidium, Gracilaria, Pterocladia and Gelidiella. Gracilaria genus is the major source of agar globally.

The nature of the agar and its properties (e.g. its gelling capacity) will vary depending on the species from which it is extracted and the extraction method used in its production, but it is envisaged that any known agar may find use in the invention. Agars obtained from Gracilaria species are typically more sulfated and therefore have a lower gelling capacity. However, their gelling properties may be enhanced by alkaline hydrolysis of the seaweed material prior to extraction. This converts the L-galactose 6-sulfate units into 3,6-anhydro-L-galactose residues which are considered to be responsible for the gelling properties of the polymer. Alternatively, pre- and/or post-extraction, agars may be subjected to enzyme treatment to remove sulfate groups.

Agars are linear polysaccharides made up of alternating β (1,3)- and α (1,4)-linked galactopyranose residues. A substantial part of the a-galactose residues may exist as the 3,6-anhydride derivative. The (1,3)-linked residue is the D-enantiomer, while the (1,4)-linked residue is the L-enantiomer. Natural chemical modifications of these structures by acidic groups (namely sulfate, uronate and pyruvate) as well as by non-ionic methoxy groups have been identified. Early studies suggested that agar consisted of two main fractions: a neutral fraction termed “agarose” having high gelling ability, and a charged fraction called “agaropectin” having a lower gelling ability. More recent studies have shown that agar is a complex mixture of polysaccharides ranging from essentially neutral to charged galactan molecules. The term “agarose” refers to the neutral polysaccharide with high gelling ability made up of repeating disaccharide units of agarobiose, i.e. 4-O-(β-D-galactopyranosyl)-3,6-anhydro-α-L-galactopyranose. The polysaccharide with repeating disaccharide units of 4-O-(β-D-galactopyranosyl)-α-L-galactopyranose in which the anyhydride bridge is absent is called “agaran”. Alkaline treatment of agar removes the sulfate ester on the C6 of the 4-linked galactose units with formation of the corresponding 3,6-anhydride form. This treatment is widely used in industrial agar extraction from Gracilaria sp. to improve its gelling properties. A more detailed overview of agar can be found, for example, in Chapter 24 of the Handbook of Hydrocolloids (Sousa et al., 2021), the entire content of which is incorporated herein by reference.

Agar is globally permitted in food products by Food Safety Authorities, including the European Food Safety Authority (EFSA) as a food additive (E-406) and the Food and Drug Administration (FDA). Agar is supplied as a powder having high solubility in water, for example at least 85% (at 80° C.). Its gel strength may vary but will typically be in the range from about 700 to about 1100 g/cm² (measured in respect of a 1.5 wt. % concentration in water at 20° C.). The gelling point of agar is typically in the range from 30 to 45° C. (measured at a 1.5 wt. % concentration in water at 20° C.). The melting point of agar may, for example, range from 80 to 95° ° C. (measured at a 1.5 wt. % concentration in water at 20° C.). Agar having a gelling point in the range from about 35 to about 45° C. (measured at a 1.5 wt. %

concentration in water at 20° C.) and/or a melting point of from about 80 to 95° C., e.g. from about 85 to 92° C. (measured at a 1.5 wt. % concentration in water at 20° C.) is particularly preferred for use in the invention.

Agar for use in the invention can be obtained from various commercial sources. Non-limiting examples of agars which may be used include Gelagar HDR 800 (from B. & V. srl, Italy), and Qsol™ High Solubility Agar and Qsol Agar (from Hispanagar, Spain).

The aqueous phase of the gelled oil-in-water emulsions according to the invention can comprise agar as the sole gelling agent, or it may comprise additional non-agar gelling agents. Where other gelling agents are present, these may be selected from other gelling agents known in the art. Consistent with the intended “vegetarian” or “vegan” nature of any of the products defined herein, any additional gelling agent should not be any animal by-product. For example, mammalian gelatin will not be present. Preferably, gelatin from any source (including fish gelatin) will not be present.

The gelling agent or combination of gelling agents will be present in the aqueous phase in an amount suitable to provide the desired degree of gelling as herein described. The amount will vary to some extent dependent on the precise nature of the gelling agent(s) (for example, the type of agar which is employed) and/or other components of the aqueous phase, but a suitable amount may readily be determined by those skilled in the art. Where a gelling agent other than agar is also employed, an appropriate amount may readily be selected by those skilled in the art. The amount of agar may be adjusted accordingly.

In one set of embodiments, agar may be present in the aqueous phase at a concentration of about 0.1 to about 7.5 wt. %, preferably about 0.25 to about 5 wt. %, particularly about 0.3 to about 3.5 wt. %, e.g. about 0.5 to about 3 wt. % (i.e. based on the weight of the aqueous phase). For example, it may be present at a concentration of 0.5, 1.0, 1.5, 2.0, 2.5 or 3.0 wt. % (based on the weight of the aqueous phase). The concentration of agar based on the overall weight of the composition may range from about 0.1 to about 5 wt. %, preferably from about 0.15 to about 4.5 wt. %, more preferably from about 0.2 to about 4 wt. %, e.g. from about 0.25 to about 3.5 wt. %, or from about 0.25 to about 3 wt. %. For example, it may be present at a concentration of 0.25, 0.50, 0.75, 1.0, 1.25, 1.50, 1.75, 2.0, 2.25, 2.5, 2.75 or 3.0 wt. % (based on the overall weight of the composition).

The gelled emulsions herein described are stabilised by a surfactant which is a plant-based protein or polysaccharide. As used herein, the term “plant-based” is intended to refer to a material that is derived (e.g. extracted) from a plant or any part of a plant, such as the fruit or seed of a plant. Such materials include derivatives of any naturally-occurring component of a plant or plant part, for example derivatives obtained by chemical modification. The plant-based surfactant for use in the invention may thus be a natural product, or it may be a semi-synthetic product.

The surfactant for use in the invention is capable of stabilising a gelled oil-in-water emulsion as herein described. In order to perform this function, it will be understood that the surfactant should be sufficiently soluble in the aqueous phase of the emulsion under the conditions used to produce the emulsion. Due to the nature of some of the surfactants proposed for use in the invention, specifically those which contain plant proteins, solubility should take into account the pH of the aqueous phase. In one embodiment, the surfactant will have a solubility of at least about 5 mg/ml in an aqueous solution at a pH of 4.5 when measured at a temperature of about 50° C. and at a pressure of about 1 atm.

The surfactant for use in the invention will be one which is suitable for use in on oral pharmaceutical or a food product. It may, for example, be any surfactant which is acceptable for use in a food product, i.e. a food grade protein, polysaccharide or any derivative thereof which is suitable for human consumption. Typically it will be a surfactant which has been approved for use as a food additive by a food-related administration (e.g. the European Food Safety Authority, or the US Food and Drug

Administration). Surfactants having an E-number and which are therefore permitted for use as food additives within the European Union are particularly suitable for use in the invention.

The plant-based surfactant for use in the invention comprises a plant protein, a plant polysaccharide, or any derivative or combination thereof. Derivatives include products obtained by chemical and/or physical modification of plant proteins, plant polysaccharides and mixtures thereof. Chemical modification may include, for example, functionalisation to introduce one or more functional groups, or hydrolysis to reduce the molecular weight of the material. Functionalisation is particularly suitable since it can be employed to adjust the hydrophobic/hydrophilic characteristics of the product. Suitable functional groups and methods for their introduction are well known in the art. Non-limiting examples of functional groups include, for example, aliphatic groups, carboxyl, amine and amide groups. Functionalisation may also involve reaction with another compound to form a conjugate, for example reaction with a glycol such as propylene glycol (“PG”).

Physical methods may include, but are not limited to, ultra-purification for example to tailor the molecular weight distribution of the material.

Plant protein derivatives which are suitable for use in the invention include, for example, hydrolysed proteins. Derivatives of plant polysaccharides for use in the invention include, for example, polysaccharides that are hydrophobically modified to impart the desired surface active properties and/or water-solubility.

The plant protein and plant polysaccharide surfactants for use in the invention are high in molecular weight and will generally be considered “macromolecular”. In one embodiment, the plant-based surfactant will have a weight average molecular weight, Mw, which is greater than or equal to about 10 kDa, for example which is greater than or equal to about 15 kDa, 20 kDa or 25 kDa. Typically, the plant-based surfactants for use in the invention will have a weight average molecular weight ranging from about 10 to about 500 kDa, for example from about 20 to about 450 kDa, or from about 25 to about 450 kDa, or from about 30 to about 450 kDa, from about 40 to about 450 kDa, from about 50 to about 450 kDa, from about 60 to about 450 kDa, or from 70 to about 450 kDa, or from 80 to about 450 kDa. In another set of embodiments, the surfactant for use in the invention may have a weight average molecular weight which ranges from about 10 to about 80 kDa, preferably from about 20 to about 70 kDa, e.g. from about 30 to about 70 kDa. Methods for the measurement of molecular weight are well known in the art. That typically used for measuring the molecular weight of any protein, for example, is SEC-MALLS (Size Exclusion Chromatography-Multiple Angle Laser Light Scattering).

Plant proteins and their derivatives having surface active properties and which are suitable for use in the invention are well known in the art. Plant proteins are typically supplied in two major forms: isolate and concentrate. Unless otherwise specified, any reference herein to “a protein” includes the protein in the form of the isolate and concentrate. Concentrates may include fat, carbohydrates and bioactive compounds, for example. Isolates are processed to remove the fat and carbohydrates and, in some cases, may also be lower in any bioactive compounds.

Plants in the legume family (Fabaceae or Leguminosae) are a significant source of proteins known for use in food products and may be used in the invention. Typically such proteins are obtained from the fruit or seed of the plant. The family Fabaceae includes, for example, Glycine max (soy bean), Phaseolus sp. (genus of beans), Pisum sativum (pea), Cicer arietinum (chickpeas), and Arachis hypogaea (peanut). Examples of legumes from which protein materials for use in the invention may be derived include, but are not limited to, peas, beans, chickpeas, lentils, soy beans (also known as soya beans) and peanuts. Other plant-based proteins which may be used in the invention include those obtained from rice, sunflower, potato, and chia, for example.

Proteins in legumes include water-soluble albumins, and salt-soluble globulin storage proteins (7S vicilin and/or 11S legumin fractions) (see, for example, Boye et al., “Pulse proteins: Processing, characterization, functional properties and applications in food and feed”—Food Research International 43(2): 414-431, 2010, the entire content of which is incorporated herein by reference). These globular proteins consist of polymorphic subunits bound together by primarily non-specific hydrophobic interactions; vicilin is a trimer, while legumin is a hexamer (see, for example, Schwenke “Reflections about the functional potential of legume proteins A Review”—Food/Nahrung 45(6): 377-381, 2001, the entire content of which is incorporated herein by reference). Legume proteins are relatively high in beta-sheet structures compared to cereal or animal protein, imparting a high structural flexibility. This aids emulsion stabilization as the proteins undergo significant conformational changes upon adsorbing to emulsion droplets, exposing hydrophobic residues to the oil phase and forming a highly stable interfacial layer (see, for example, Tang et al., “A comparative study of physicochemical and conformational properties in three vicilins from Phaseolus legumes: Implications for the structure-function relationship”—Food Hydrocolloids 25(3): 315-324, 2011; and Sharif et al., “Current progress in the utilization of native and modified legume proteins as emulsifiers and encapsulants—A review”—Food Hydrocolloids 76: 2-16, 2018, the entire contents of which are incorporated herein by reference).

Particularly suitable for use in the invention are proteins obtained from peas or beans, including isolates and concentrates of such proteins. Those from soy bean and faba bean are particularly suitable.

Pea and bean protein isolates are highly refined or purified forms of pea and bean protein. Pea protein isolate typically has a minimum protein content of about 80% (dry basis), whereas bean protein isolate may have a minimum protein content of about 65%, sometimes as high as 90% (dry basis). Pea protein can be obtained from a variety of species of pea. Bean protein can be obtained from a variety of species of bean including, but not limited to, faba bean and soy bean. Soy protein isolates are a highly refined or purified form of soy protein with a minimum protein content of about 90% (dry basis). Soy protein isolates are made from defatted soy flour from which most of the non-protein components, such as fats and carbohydrates, have been removed. Pea and bean proteins, including isolates and concentrates, are suitable for vegetarian and vegan diets. Commercial sources of pea protein include Nutralys and Hill Pharma. Commercial sources of bean protein, for example, faba bean protein include Vestkorn and Hill Pharma. Commercial sources of soy protein include PHH (Supro 590).

Plant polysaccharides and their derivatives having surface active properties and that are suitable for use as surfactants in the invention are well known in the art. These include, for example, celluloses, starches, alginates and derivatives thereof.

In many cases, these materials will be chemically modified to impart the required surface active properties and/or to provide the desired degree of water solubility.

In one embodiment, the polysaccharide for use in the invention will be a hydrophobically-modified polysaccharide. A “hydrophobically-modified polysaccharide” means a polysaccharide that incorporates one or more hydrophobic groups. Typically, such a material will be produced by reacting a portion of the side-chains along the polymer backbone with at least one hydrophobic group. Such hydrophobic groups include, for example, alkyl, alkenyl, cycloalkyl, aryl and arylalkyl groups. The alkyl and alkenyl groups may be straight-chained or branched. The hydrophobic groups may contain up to about 22 carbon atoms. In some cases, such groups may be short chain alkyl groups, for example C₁₋₆ or C₁₋₃ alkyl groups. Methyl, ethyl and propyl groups are particularly suitable.

Natural cellulose materials are typically not water-soluble. Although they contain many hydroxyl groups, these form strong intermolecular hydrogen bonds which prevent the access of water molecules. Chemical modification of the cellulose to replace some of the hydrogen atoms of the hydroxyl groups by substituents such as methyl groups (—CH₃), hydroxypropyl groups (—CH₂CHOHCH₃), or hydroxyethyl groups (—CH₂CH₂OH) interrupts the intermolecular hydrogen bonding to render the cellulose water-soluble. Examples of modified cellulose materials which are suitable for use in the invention include methyl cellulose (MC), hydroxypropyl methyl cellulose (HPMC) and carboxymethyl cellulose (CMC).

Modified starches which may be used as surfactants in the invention include acetylated starch, hydroxypropyl starch, hydroxy propyl distarch phosphate, starch sodium octenyl succinate, and acetylated oxidised starch. Specific examples of suitable starches include the following food grade starches: E1401 Modified starch; E1402 Alkaline modified starch; E1403 Bleached starch; E1404 Oxidised starch; E1410 Monostarch phosphate; E1412 Distarch phosphate; E1413 Phosphated distarch phosphate; E1414 Acetylated distarch phosphate; E1420 Acetylated starch, mono starch acetate; E1422 Acetylated distarch adipate; E1430 Distarch glycerine; E1440 Hydroxy propyl starch; E1441 Hydroxy propyl starch; E1442 Hydroxy propyl distarch phosphate; E1450 Starch sodium octenyl succinate; and E1451 Acetylated oxidised starch. Preferred for use in the invention are starches having the following E-numbers: E1414, E1420, E1422, E1440, E1441, E1442, E1450 and E1451.

Alginates that are suitable for use as surfactants in the invention are those which have been hydrophobically modified. A chemically modified alginate which may be used in the invention is Propylene Glycol Alginate (PGA). PGA is an ester of alginic acid in which some of the carboxyl groups are esterified with propylene glycol, some are neutralized with an appropriate alkali and some remain free. PGA is available under the E-number E405.

In certain embodiments, the plant-based surfactant for use in the invention may comprise a combination of a protein and a polysaccharide. For example, it may be a polysaccharide-protein complex or conjugate. Alternatively, it may comprise a mixture of a protein and a polysaccharide.

Plant gum exudates are also suitable for use as surfactants in the invention and include, for example, Gum Arabic and Gum Ghatti. Gum Arabic is a substance obtained from two sub-Saharan species of the Acacia tree, Acacia senegal and Acacia seyal. It is widely used in the food industry under the E-number E-414. It is a complex mixture of glycoproteins and polysaccharides predominantly consisting of arabinose and galactose. Gum Ghatti is the dried exudate of the Anogeissus latifolia tree and is a complex, water soluble polysaccharide.

Any of the plant-based surfactants herein described may be used in combination.

In one embodiment of the invention, for example, a plant-based protein or derivative thereof as herein described may be used in combination with Gum Arabic. A preferred combination is a pea or bean protein, protein isolate or protein concentrate and a plant gum exudate (e.g. Gum Arabic), for example a combination of faba bean protein and Gum Arabic.

The surfactant (or combination of surfactants) is present in an amount effective to provide the desired stability to the emulsion. The amount will vary dependent on factors such as the precise nature of the surfactant(s), the relative proportions of the oil and aqueous phase, and the presence (and amount) of any other components of the emulsion that may act as an emulsifying agent. Taking account of these factors, an appropriate amount of the plant-based surfactant(s) may readily be determined by those skilled in the art. A suitable amount may, for example, be in the range from 0.1 to 5.0 wt. %, preferably from 0.25 to 4.0 wt. %, particularly from 0.5 to 3.0 wt. %, e.g. from 1.0 to 2.5 wt. % (based on the total weight of the overall composition). For example, the amount of the surfactant(s) may be 1.0, 1.25, 1.5, 1.75, 2.0, 2.25 or 2.5 wt. %, based on the total weight of the composition. When a combination of surfactants is used, their relative amounts may readily be selected by those skilled in the art.

When employing a surfactant that comprises a combination of a plant-based protein or derivative thereof, such as a pea or bean protein, protein isolate or protein concentrate (e.g. faba bean protein), and a plant gum exudate (e.g. Gum Arabic), each component may be present in an amount in the range from 0.5 to 2.0 wt. %, preferably 1.0 to 1.5 wt. % (based on the total weight of the composition). For example, a surfactant comprising 1.0 to 2.0 wt. % faba bean protein and 0.5 to 1.5 wt. % Gum Arabic may be particularly suitable.

In one set of embodiments, glycerol may be present in the aqueous phase of the emulsion. Advantageously, glycerol may be present in an amount effective to reduce the water activity of the composition and thus reduce microbial growth. Water activity may, for example, be reduced to below about 0.8, for example in the range 0.5 to 0.8, or 0.6 to 0.75, or 0.65 to 0.75. A proportion of the water in the aqueous phase of the emulsion may, for example, be replaced by glycerol. For example, up to 90 wt. % of the water may be replaced by glycerol. In other embodiments, from 10 to 90 wt. %, preferably from 50 to 85 wt. %, e.g. from 55 to 75 wt. % of the water may be replaced by glycerol. When glycerol is present, this can reduce the amount of any preservative agent that may be required to provide a product having an adequate shelf-life. In some cases, it may avoid the need for any preservative agent to be present. As herein described, the presence of sugar alcohols in the aqueous phase also contributes to a reduction in water activity. The amount of glycerol may be adjusted taking into account the amount of any sugar alcohols that may be present. In some embodiments, glycerol may replace the sugar alcohols, or the presence of glycerol may reduce the amount of sugar alcohols.

If present, glycerol may be provided in an amount of up to 60 wt. %, preferably from 20 to 60 wt. %, for example from 30 to 60 wt. % based on the total weight of the composition.

The oil phase of the emulsion will comprise a physiologically tolerable lipid, or a mixture of different physiologically tolerable lipids. Depending on the nature of the lipid (or lipids), the oil phase itself may have nutraceutical and/or pharmaceutical properties. In some embodiments, therefore, the lipids which constitute the oil phase of the emulsion may be the nutraceutical or pharmaceutical agent. Examples of such lipids include, for example, essential fatty acids such as those which are herein described. Alternatively, the oil phase may act as a carrier for a lipophilic pharmaceutical or nutraceutical agent. In this case, the active agent may be dissolved or dispersed in the oil phase.

A range of different lipids are known for oral use in pharmaceutical and/or nutraceutical products and any of these may be used in the oil phase of the emulsions herein described. Sources of lipids include plant oils, such as but not limited to, rapeseed oil, sunflower oil, corn oil, olive oil, sesame oil, palm kernel oil, coconut oil, nut oils (e.g. almond oil or peanut oil), algal oil and hemp oil. Fish oils and lipids obtained from fish oils are also suitable for use in certain compositions according to the invention. Compositions containing these products are acceptable to pescetarians, for example.

Lipids derived from natural products typically comprise a mixture of different lipid components. In one embodiment, the oil phase will thus comprise a mixture of different lipids. For example, it may comprise a mixture of lipids having different chain lengths and/or different degrees of saturation.

Lipids for use in the invention may be liquid, solid or semi-solid at ambient temperature (i.e. at temperatures of about 18° C. to about 25° C.). Those which are liquid at such temperatures are generally preferred. Any combination of liquid, solid and semi-solid lipids may also be used. Solid lipids having a melting point below about 100° C., preferably below about 70° C., e.g. below about 50° C. may be used in the invention. Solid lipids which may be used include butter, solid coconut fraction, cocoa butter or cocoa fat, etc. If desired, the overall melting point of the lipids which make up the oil phase may be modified by mixing different lipids, for example by mixing a solid lipid (e.g. butter) with a liquid oil. An overall melting point in the range from 45 to 50° C. may be desirable.

Lipids for use in the invention include, in particular, fatty acids and their derivatives. These include both naturally occurring fatty acids and their derivatives, as well as synthetic analogues. In one embodiment, the oil phase may comprise a mixture of different fatty acids, or fatty acid derivatives.

The hydrocarbon chain of the fatty acid or fatty acid derivative may be saturated or unsaturated, and it may be un-branched or branched. Preferably, it will be un-branched. Typically the hydrocarbon chain will comprise from 4 to 28 carbon atoms, and generally it will have an even number of carbons. Fatty acids differ in their chain length and may be categorized as “short”, “medium”, “long”, or “very long” chain fatty acids. Those having a hydrocarbon chain of 5 or fewer carbons are referred to as “short-chain fatty acids”; those with a hydrocarbon chain of 6 to 12 carbon atoms are referred to as “medium-chain fatty acids”; those with a hydrocarbon chain of 13 to 21 carbons are referred to as “long-chain fatty acids”;

and those with a hydrocarbon chain of 22 carbons or more are referred to as “very long-chain fatty acids”. Any of these may be used in the invention.

In one embodiment, the oil phase will comprise a saturated fatty acid, or a derivative of a saturated fatty acid including, but not limited to, any of the derivatives herein described. Medium-chain saturated fatty acids and their derivatives find particular use in the invention. Those having from 8 to 12, e.g. 8, 10 or 12, carbon atoms in the hydrocarbon chain are particularly preferred—i.e. caprylic acid (C8), capric acid (C10) or lauric acid (C12), and any derivatives thereof. Typically, a saturated fatty acid or derivative thereof may be used as a carrier for one or more active components in the oil phase, for example as a carrier for a pharmaceutical or nutraceutical agent.

Saturated fatty acids and their derivatives for use in the invention may be naturally occurring or they may be synthetically produced. Most typically, they will be naturally occurring and thus may be used in the form of mixtures of different fatty acids and/or different fatty acid derivatives. Sources of saturated fatty acids and their derivatives include, for example, coconut oil and palm kernel oil.

In another embodiment, the oil phase may comprise an unsaturated fatty acid or derivative thereof in which the carbon chain contains one or more carbon-carbon double bonds. The double bonds may be in the cis- or trans-configuration, or any combination thereof where more than one double bond is present. Those in which the double bonds are present in the trans-configuration are generally less preferred due to the need to reduce the consumption of so-called “trans-fats” as part of a healthy diet. Fatty acids and their derivatives having cis-configuration double bonds are thus preferred. Mono- and poly-unsaturated fatty acids and their derivatives are well known in the art. Such fatty acids typically will contain 12 to 26 carbons, more typically 16 to 22 carbons, and will have a mono- or poly-unsaturated hydrocarbon chain. They include, in particular, the polyunsaturated fatty acids (PUFAs) such as the essential fatty acids.

Oils which contain long chain, unsaturated fatty acids and their derivatives find particular use in the invention, for example in any composition which is intended for use as a nutraceutical. Particularly important essential fatty acids which may be used include the ω-3, ω-6 and ω-9 fatty acids. Examples of ω-3 fatty acids include alpha-linolenic acid (ALA), stearidonic acid (SDA), eicosatrienoic acid (ETE), eicosatetraenoic acid (ETA), eicosapentaenoic acid (EPA), docosapentaenoic acid (DPA), docosahexaenoic acid (DHA), tetracosapentaenoic acid and tetracosahexaenoic acid. Examples of ω-6 fatty acids include linoleic acid, gamma-linolenic acid, eicosadienoic acid, dihomo-gamma-linolenic acid (DGLA), arachidonic acid (AA), docosadienoic acid, adrenic acid, docosapentaenoic acid, and calendic acid. Examples of ω-9 fatty acids include oleic acid, eicosenoic acid, mead acid, erucic acid and nervonic acid.

Sources of unsaturated fatty acids and their derivatives include oils obtained from various fish, plant, algae, and microorganism sources. Particularly suitable sources are algae oils and plant oils, however fish oils may also be suitable for those that follow a pescetarian diet. These oils are all rich in ω-3, ω-6 and ω-9 fatty acids. Fish oils may, for example, be obtained from anchovies, sardines and mackerel.

Any known derivatives of the fatty acids may be used in the invention. These include, in particular, the carboxylic esters, carboxylic anhydrides, glycerides (i.e. mono-, di-, or triglycerides) and phospholipids. As used herein the term “derivatives” in the context of a fatty acid also encompasses any pharmaceutically acceptable salt of a fatty acid. Suitable salts are well known to those skilled in the art and include, but are not limited to, the lithium, sodium, potassium, ammonium, meglumine, and diethylamine salts.

Examples of carboxylic acid esters of fatty acids include compounds having a terminal —CO₂R group in which R is a straight-chained or branched alkyl group, typically a short chain alkyl, preferably a C₁₋₆ alkyl group, e.g. selected from methyl, ethyl, n-propyl, iso-propyl, n-butyl, sec-butyl and n-hexyl.

Where the fatty acid derivative is a carboxylic anhydride, it may include a terminal —CO₂COR group in which R is a straight-chained or branched alkyl group, typically a short chain alkyl, preferably a C₁₋₆ alkyl group, e.g. selected from methyl, ethyl, n-propyl, iso-propyl, n-butyl, sec-butyl and n-hexyl.

Glycerides are esters derived from glycerol and up to three fatty acids. The fatty acids present may be any of those herein described and thus they may be saturated or unsaturated, for example. In the case of di- and tri-glycerides the fatty acid components may be the same or different. For example, these may be of different chain lengths.

In one embodiment, the lipid carrier for use in the invention may comprise a medium chain triglyceride (MCT). MCTs are triglycerides with two or three medium-chain fatty acids which may be identical or different. Sources of MCTs include coconut oil and palm kernel oil, for example. The fatty acids present in MCTs are typically saturated medium chain fatty acids. MCTs from coconut oil, for example, comprise C₆₋₁₂ fatty acids, predominantly C₈ and C₁₀ fatty acids. A typical fatty acid composition of an MCT oil obtained from coconut oil may, for example, comprise: 0.1 wt. % caproic acid (C 6:0), 55 wt. % caprylic acid (C 8:0), 44.8 wt. % capric acid (C 10:0), and 0.1 wt. % lauric acid (C 12:0).

Phospholipids generally consist of a glycerol molecule linked to two fatty acids (the “tail” groups) and to a hydrophilic “head” group which consists of a phosphate group. The phosphate group may be modified by linkage to choline, ethanolamine or serine. In one embodiment, the oil phase may be constituted in whole or part by a phospholipid, for example a plant lecithin.

The amount of oil present in the compositions of the invention will be dependent on factors such as the nature of the oil, the nature and desired loading level of any pharmaceutical or nutraceutical that may be present, etc. and can be varied according to need. The oil phase may, for example, constitute from 5 to 50 wt. %, preferably from 10 to 45 wt. %, for example from 15 to 40 wt. %, from 15 to 30 wt. % or from 20 to 25 wt. % of the gelled oil-in-water emulsion.

As will be understood, in the compositions of the invention the oil provides the discontinuous phase within a continuous aqueous phase which is gelled. The oil is thus dispersed throughout the gelled aqueous phase in the form of oil droplets (also referred to herein as oil “particles”). Gelling of the aqueous phase provides a stable emulsion which prevents coalescence of the droplets of oil, for example due to the prevention of physical collisions between droplets. When preparing the gelled emulsions, the plant-based surfactant will initially be dissolved in the aqueous phase but migrates to the oil-water interface where it serves to stabilise the oil phase. Although not wishing to be bound by theory, it is believed that an uneven distribution of the large molecules of the surfactant around the oil droplets serves to provide a friction layer which provides a “semi active filler” effect.

The size of the oil particles in the gelled oil-in-water emulsion is not particularly limited. For example, oil particles having a volume-based size in the range from about 100 nm to about 100 μm, preferably from about 500 nm to about 75 μm, in particular from about 750 nm to about 50μm, e.g. from about 1000 nm to about 40 μm may be provided. Volume-based average particle sizes may range from about 5 to 50 μm, preferably from about 5 to 30 μm, e.g. about 5 to 25 μm. “Volume-based average” as used herein refers to the volume moment mean or De Brouckere Mean Diameter (also known as the “D[4,3]” value). This reflects the size of those particles which constitute the bulk of the sample volume and is most sensitive to the presence of large particles in the size distribution.

An essentially homogenous size distribution of oil particles may be desirable. The D₉₀ value indicates the size value which 90% of the oil particles meet out of the entirety of all of the oil droplets. D₉₀ values may range from 15 to 80 μm, preferably 25 to 65 μm, in particular from 30 to 50 μm. Correspondingly, the D₅₀ and D₁₀ value, respectively, indicate the size value which 50% and 10% of the oil droplets meet out of the entirety of all of the oil droplets. D₅₀ values may range from 10 to 45 μm, in particular from 15 to 35 m, e.g. from 18 to 25 μm. D₁₀ values may range from 0.5 to 20 μm, in particular from 3 to 15 μm, e.g. from 5 to 10 μm.

Lipid droplet size and size distributions can be determined using methods and apparatus conventional in the art, for example using a Malvern Mastersizer 3000 (Worcestershire, UK) connected to a Hydro MV, wet dispersion unit (Malvern, Worcestershire, UK). Analysis of the data may be performed using the manufacturer's software (Mastersizer 3000, v1.0.1). Testing may be carried out by dissolving and diluting the gelled emulsion in a suitable solvent (1:100) at 50° C.

Suitable solvents include Milli-Q water and a 10% (v/v) HCl solution (the latter may minimize flocculation during testing). The refractive index of water and corn oil is set to 1.33 (solvent) and 1.47 (dispersed phase), respectively, and the absorption index of the dispersed droplets set to 0.01. To avoid multiple scattering or low intensity of the scattered light, the dissolved emulsion is added to the dispersion unit (containing ˜125 mL water), until an obscuration of approximately 10% is obtained.

The size and size distribution of the oil particles may be varied. If desired, size reduction of the oil particles can be achieved by various different means, for example by mechanical processes or by chemical processes involving the selection of smaller lipid molecules, or indeed by a combination of these approaches. Chemical methods suitable for achieving a size reduction of the oil particles may involve the selection of a particular type of lipid (or combination of lipids) capable of forming smaller oil droplets. Certain oils, such as MCTs for example have a tendency to produce a finer dispersion of oil droplets. Mechanical reduction involves the use of shear forces to break down larger oil droplets into smaller nano-scale particles. Smaller particles may thus be produced by suitable adjustments to the method used to produce the emulsion, for example by varying the shear force and/or the duration of mixing of the oil and aqueous phases. The use of higher shear forces and/or longer mixing times will produce smaller particles of oil. Suitable shear may be achieved, for example, using a conventional homogenizer such as a rotor-stator mixer, e.g. an Ultra-turrax® homogenizer. A problem often encountered in mechanical processes for the production of oil-in-water emulsions is the re-aggregation (i.e. coalescence) of the particles, but this is addressed in the invention by the use of a gelled aqueous phase which serves to stabilize the emulsion and the use of a surfactant which reduces the energy required for emulsification (by reducing interfacial tension) and which protects the droplets against re-aggregation.

The aqueous phase (i.e. continuous phase) of the gelled oil-in-water emulsion may constitute from 50 to 95 wt. %, preferably from 55 to 90 wt. %, for example from 60 to 85 wt. %, from 70 to 85 wt. %, or from 75 to 80 wt. % of the composition.

In addition to water, the gelling agent(s) and the surfactant(s), other physiologically tolerable materials may also be present in the aqueous phase, for example, pH modifiers (e.g. buffering agents), viscosity modifiers (e.g. thickening agents, plasticizers), sweeteners, bulking agents (i.e. fillers), anti-oxidants, aromas, flavouring agents, and colouring agents. The nature and concentration of any such materials may readily be determined by those skilled in the art.

The presence of bulking agents (i.e. fillers) in the aqueous phase aids in reducing water activity and thus in reducing microbial growth. Water activity may, for example, be reduced to below about 0.8, for example in the range 0.5 to 0.8, or 0.6 to 0.75, or 0.65 to 0.75. The amount and type of bulking agents may readily be selected by those skilled in the art. Suitable examples include, but are not limited to, sugar alcohols, sugars and mixtures thereof. Suitable sugar alcohols include sorbitol and xylitol and mixtures thereof. Sugars which may be used include trehalose, sucrose, glycerol and mixtures thereof. Bulking agents may constitute from 45 to 70 wt. %, preferably 50 to 65 wt. %, e.g. 55 to 60 wt. %, based on the aqueous phase. In some cases, the selected bulking agent(s) may also act as sweetening agents depending on their concentration. For example, the compositions according to the invention may contain xylitol, e.g. as 0.5 to 50 wt. %, preferably 1 to 40 wt. %, e.g. 15 to 40 wt. %, in order to improve taste.

Where a sweetener is included in the aqueous phase, this will typically be selected from natural sweeteners such as sucrose, fructose, glucose, reduced glucose, maltose, xylitol, maltitol, sorbitol, mannitol, lactitol, isomalt, erythritol, polyglycitol, polyglucitol, glycerol and stevia, and artificial sweeteners such as aspartame, acesulfame-K, neotame, saccharine, and sucralose. The use of non-cariogenic sweeteners is preferred.

In one embodiment, viscosity modifiers may also be provided in the aqueous phase. Suitable viscosity modifiers include other hydrocolloids such as starch, modified starch (e.g. hydroxy ethyl starch, hydroxy propyl starch), xanthan, galactomannans (e.g. guar gum and locust bean gum), gum karaya, gum tragacanth, and any combination thereof. As will be understood, a viscosity modifier may possess some surface-active properties and may additionally aid in stabilisation of the emulsion. The thickening effect of the viscosity modifier depends on the type of material (e.g. hydrocolloid) used and its concentration, the other components and the pH of the formulation, etc. but suitable amounts may readily be determined by those skilled in the art. Typical amounts of any viscosity modifier which may be present may range from 0.1 to 5 wt. % of the overall composition, preferably from 0.2 to 2.5 wt. %, for example from 0.5 to 2.0 wt. %.

Flavoring agents may be present in the compositions and may, for example, aid in taste masking certain lipids such as those which contain omega-3 fatty acids. Suitable flavors include, but are not limited to, citrus flavors, for example orange, lemon or lime oil.

pH modifiers may readily be selected by those skilled in the art and include food grade acids such as citric acid. Buffering agents may also be used to adjust pH and include organic acid/base buffering systems. Suitable buffering agents are well known in the art and include, for example, sodium citrate and malic acid, etc. The pH of the aqueous phase of the emulsion may be adjusted to be in the range from 2 to 8, particularly 3 to 7, preferably 3.5 to 6, for example 4 to 5.

Where antioxidants are present in the aqueous phase these will be water soluble and include, for example, ascorbic acid, citric acid and salts thereof such as sodium ascorbate. Depending on the choice of oil, these may be supplied in a form which contains an antioxidant such as vitamin E, for example. If present, the amount of any anti-oxidant(s) may be up to 3 wt. % of the overall formulation, e.g. up to 1 wt. %.

In addition to the lipid(s), the oil phase of the emulsion may also if desired contain physiologically tolerable lipid soluble materials, for example pharmaceutically acceptable agents, anti-oxidants (e.g. vitamin E), flavorings, and coloring agents.

In some embodiments, additional physiologically active agents may also be present in the gelled emulsions herein described. These may be provided in the aqueous and/or oil phases and may be dissolved and/or dispersed in one or both of these phases. Other actives which may be present in the oil phase include fat soluble active agents.

In one embodiment, the gelled oil-in-water emulsions according to the invention may comprise, consist essentially of, or consist of, the following components:
(a) water;
(b) at least one plant-based surfactant;
(c) one or more physiologically tolerable lipids;
(d) agar;
(e) one or more bulking agents;
(f) optionally one or more pH modifiers;
(g) optionally one or more viscosity modifying agents (e.g. thickening agents or plasticisers); and
(h) optionally one or more additional physiologically active agents.

By “consisting essentially of” it is intended that the emulsions will be substantially free from (e.g. free from) other components which materially affect their properties.

By “consists of” it is intended that the emulsions will be substantially free (e.g. free from) from any other components than those listed.

In one set of embodiments the compositions of the invention may be provided in the form of a dose unit. By “dose unit” it is intended that the composition will be taken orally by the subject (e.g. administered to a patient) “as received”, i.e. it will not be broken or cut before oral delivery. The weight of the dose unit will therefore be such that the composition is suitable for delivery in this way. For example, it may have an overall weight in the range from 50 to 3,000 mg, e.g. 250 to 3,000 mg or 500 to 2,500 mg, especially 100 to 2,000 mg, e.g. 750 to 2,000 mg, particularly 100 to 1,500 mg, more particularly 400 to 1,500 mg, more especially 400 to 1,000 mg. In one set of embodiments, the dose units will generally be quite large, e.g. having a mass of from 400 to 3,000 mg, e.g. 600 to 1,500 mg. The overall dose unit weight may be selected as required. For example, it may be scaled up or down dependent on the nature of the selected active components and their intended dose.

Each dose unit will typically consist of a self-supporting, gelled oil-in-water emulsion as herein described. As will be understood, in this case the dose unit will contain only the defined oil and aqueous phases, i.e. it will be free from any other components. Individual dose units may be prepared from a larger piece of the gelled emulsion which is divided, e.g. by cutting. More typically, however, each dose unit will be formed by extrusion or moulding from a liquid emulsion, or incompletely gelled emulsion, prior to gelation (i.e. above the gelling temperature of the gelling agent).

Alternatively, a core of the gelled oil-in-water emulsion may be provided with a suitable coating of a physiologically tolerable coating material. Such coatings may be of the type conventional in the pharmaceutical and nutraceutical industry and may be applied by any conventional means, for example by dipping or spraying. In one set of embodiments, the gelled oil-in-water emulsions herein described may therefore be provided with a coating. For example, these may be provided within a capsule shell which dissolves in the mouth. Viewed from another aspect the invention thus provides an orally administrable capsule comprising a capsule shell enclosing a gelled oil-in-water emulsion as herein described.

In the capsules of the invention, the shell may be of any physiologically tolerable material but will typically be a sugar, a biopolymer or a synthetic or semi-synthetic polymer which is soluble or disintegrable in saliva or fluid within the gastrointestinal tract. The shell may be soft, but is preferably substantially rigid. Particularly desirably, the capsules will have the consistency of a “jelly bean”. The shell will preferably be of a material and a thickness to prevent oxidation of the contents. The shell may comprise a sugar or cellulose, for example sorbitol. The use of sugars and cellulose as capsule shell materials is well-known in the pharmaceutical and nutraceutical fields.

The capsule shell material may thus typically be a sugar, e.g. sucrose, fructose, maltose, xylitol, maltitol or sorbitol, but may additionally contain hydrocolloid materials such as for example carageenan, alginate, pectin, cellulose, modified cellulose, starch, modified starch, gum arabic, etc. The capsule shell may contain other ingredients such as, for example, artificial sweeteners, colors, fillers, flavors, antioxidants, etc.

The capsule shell may be pre-formed such that the oil-in-water emulsion can be filled into the shell either as a liquid, or once set. Alternatively a shell precursor (e.g. a solution) may be coated onto the set emulsion, for example using standard coating techniques. If desired the capsule may be further coated, e.g. with a wax.

Preparation of the gelled oil-in-water emulsions herein described may be carried out by emulsification of the aqueous and oil phase components. It will be understood that emulsification is carried out under conditions in which the aqueous phase is a liquid (for example a viscous liquid), i.e. prior to the formation of a gel. Emulsification will thus be carried out at a temperature above the sol-gel transition temperature of the agar gelling agent. Subsequent cooling of the emulsion below its sol-gel temperature results in the desired gelled emulsion.

Prior to emulsification, any selected active agents may be added to the oil and/or aqueous phase of the composition. This may be done, for example, by dissolving the active in the selected oil or in the aqueous phase prior to forming the emulsion. Alternatively, the selected active agent(s) may be added to a mixture of the aqueous and oil phase components prior to emulsification. During the emulsification process, the active agents will typically migrate to the oil or aqueous phase depending on their hydrophilic/lipophilic characteristics.

Emulsion formation may be effected by conventional techniques and using known equipment, for example a homogenizer based on the rotor-stator principle. The speed and duration of stirring may be adjusted as required, for example it may be varied to achieve the desired shearing force to provide the desired droplet size.

Emulsification will generally be carried out under a controlled atmosphere in order to avoid oxidative degradation of the lipid and/or any active agents. For example, emulsification may be carried out in the presence of a non-oxidising gas such as nitrogen. De-gassing to remove air bubbles may also be carried during the production process, for example prior to mixing the components of the emulsion, once the liquid emulsion has been formed, prior to packaging of the set emulsion, etc. De-gassing may be carried out using any conventional means such as the application of a vacuum, or sparging with a non-oxidising gas (e.g. nitrogen). After emulsification and gelling, the emulsion may be dried to reduce the water content. If dried, however, it will still retain a continuous gelled aqueous phase as herein described and a water content within the limits herein defined.

The gelled oil-in-water emulsions will typically be provided in dose unit form as herein described. Individual dose units may be formed by methods such as molding, extrusion or cutting. Typically, however, the dose units may be formed by filling of the liquid emulsion into molds, e.g. the individual molds of a blister pack which is then sealed. The dose units will typically be in tablet or lozenge form.

Methods for preparation of the gelled oil-in-water emulsions herein described form a further aspect of the invention. Viewed from a further aspect, the invention thus provides a method for preparing an orally administrable, gelled oil-in-water emulsion, said method comprising: forming an oil phase which comprises one or more physiologically tolerable lipids; forming an aqueous phase comprising a gelling agent which is agar; combining said oil phase and said aqueous phase to form an oil-in-water emulsion in the presence of a plant-based surfactant as herein described; and allowing said emulsion to gel. Optionally, prior to or after allowing the emulsion to gel, the emulsion may be divided into individual dose units.

The dose units are preferably individually packaged in air-tight containers, e.g. a sealed wrapper or more preferably a blister of a blister pack. In another aspect, the invention thus provides a package comprising an air-tight and light-tight compartment containing one dose unit of a composition according to the invention. By excluding both air (i.e. oxygen) and light from the packaged dose unit, long term stability of the active components is enhanced.

The packages according to the invention are preferably provided in the form of blister packs containing at least two dose units, e.g. 2 to 100, preferably 6 to 30 dose units. The blister pack will generally comprise a metal, metal/plastic laminate or plastic sheet base having molded indentations in which the dosage form is placed. The pack is normally sealed with a foil, generally a metal or a metal/plastic laminate foil, for example by applying heat and/or pressure to the areas between the indentations. The use of a metal or metal/plastic laminate to form the blister pack serves to prevent air (i.e. oxygen), light and humidity from penetrating the contents of the blister pack thus enhancing the stability of active component(s). The packages according to the invention are preferably filled under a non-oxidising gas atmosphere (e.g. nitrogen) or are flushed with such a gas before sealing.

The use of agar as a gelling agent in the compositions according to the invention provides additional advantages in relation to packaging of individual dose units in a blister pack and their removal by the end user. When an emulsion in liquid form is used to fill the indentation of the blister pack (i.e. prior to gelling), it will be in intimate contact with the inner surface of the indentation. After setting of the dose unit and sealing of the blister pack, it is important that the dose unit can easily be removed from the blister pack. The presence of gelatin, first developed as a glue, in known gelatin-based compositions can give rise to the difficulty in removing these from certain surfaces, such as those made from plastic materials, especially when a liquid emulsion containing the gelatin has been allowed to set in contact with the surface. In such cases, once set, the dose unit tends to adhere to the surface and must be torn away often causing the dose unit to fragment in the process which is not acceptable. When packaging any conventional gelatin-based dose unit, it is necessary for the internal surface of a blister pack to be coated with a suitable release agent such as a neutral oil or fat. Specially developed blister pack materials having release agents incorporated onto their internal surfaces are available but add to the cost of the packaging process. The use of a release agent also leads to a surface coating of the agent on the dose unit once it has been removed from the blister pack and this can give rise to an unpleasant feel or taste of the product.

In contrast to the use of gelatin, agar-based dose units do not adhere to conventional blister pack materials. This means that standard materials can be used, including metal/plastic laminate or a plastic film over which a plastic/metal foil laminate is heat sealed. Suitable blister trays with pre-formed cavities may, for example, be formed from laminated materials such as Tekniflex® Aclar® VA10600 (TekniPlex), Perlalux® (Perlen Packaging), Formpack® (Amcor), and Regula® (Constantia Flexibles). Such materials do not have any surface coating containing a release agent.

In one embodiment, the dose units of the invention may thus be packaged in a blister pack having an internal surface which is not coated with any release agent.

Such blister packs containing a dose unit as herein described form a further aspect of the invention.

The gelled oil-in-water emulsions according to the invention find use both as pharmaceuticals, i.e. for therapeutic purposes, and as nutraceuticals to maintain or augment health and/or general well-being of a human or animal subject. For this purpose, it is intended that they are taken orally, lightly chewed in the mouth and then swallowed. It is not intended that they should remain in the mouth or need to be chewed for an extended period. Due to their soft texture, light chewing is sufficient to fragment the dosage form into smaller pieces which are easily swallowed. What the Applicant has surprisingly found is that the gelled oil-in-water emulsions according to the invention have a much better mouthfeel than pure aqueous agar gels. Whereas aqueous agar gels are brittle and ‘fracture’ in the mouth on chewing, the emulsions herein described have a greater resistance to deformation when chewed and are less susceptible to fracturing. This provides a much more acceptable chewing experience for the patient or consumer.

When used as nutraceuticals, for example, the compositions herein described may be used as a supplement (e.g. as a dietary supplement) for maintaining the general health and/or well-being of a subject. Any agent known for its nutraceutical effects may be provided in the compositions and suitable agents are well known in the art. Suitable nutraceuticals include, but are not limited to, any of the following: essential fatty acids (e.g. mono and poly-unsaturated fatty acids), essential amino acids (e.g. taurine, tryptophan, tyrosine, cysteine and homocysteine), vitamins (e.g. vitamins A, B1-B12, C, D, E, K and folate), minerals (e.g. iodine, selenium, iron, zinc, calcium and magnesium), flavonoids, carotenoids (e.g. beta carotene, alpha carotene, luteine, zeoxantaine, xanthophylls and lycopene), phytosterols, sapponins, probiotics, dietary fibres (e.g. insoluble fibre and beta-glucans), and plant extracts (e.g. aloe vera, evening primrose oil, garlic, ginger, ginseng, green tea, caffeine and cannabinoids). Where magnesium or calcium are present, these will generally be used in the form of their phosphate salts.

In particular, the gelled oil-in-water emulsions herein described may be used as a source of one or more essential fatty acids, such as PUFAs or their esters, e.g. omega-3, omega-6 and/or omega-9 fatty acids and their ester derivatives.

Examples of omega-3 acids include a-linolenic acid (ALA), stearidonic acid (SDA), eicosatrienoic acid (ETE), eicosatetraenoic acid (ETA), eicosapentaenoic acid (EPA), docosapentaenoic acid (DPA), docosahexaenoic acid (DHA), tetracosapentaenoic acid and tetracosahexaenoic acid. Examples of omega-6 acids include linoleic acid, gamma-linolenic acid, eicosadienoic acid, dihomo-gamma-linolenic acid (DGLA), arachidonic acid (AA), docosadienoic acid, adrenic acid, docosapentaenoic acid, and calendic acid. Examples of omega-9 acids include oleic acid, eicosenoic acid, mead acid, erucic acid and nervonic acid. Omega-3 acids are especially preferred, particularly EPA and DHA.

The health benefits of essential fatty acids, in particular omega-3 fatty acids, are well known. For example, these may lower triglyceride levels and/or lower cholesterol levels. Omega-3 fatty acids are vital to everyday life and health. The beneficial effects of EPA and DHA on lowering serum triglycerides are well known. They are also known for other health benefits such as cardio-protective effects, e.g. in preventing cardiac arrhythmias, stabilising atherosclerotic plaques, reducing platelet aggregation, and reducing blood pressure. They find use therefore in treating and/or preventing vascular disease. Other benefits of omega-3 fatty acids include the prevention and/or treatment of inflammation and neurodegenerative diseases, and improved cognitive development and function.

The essential fatty acids may form part or the whole of the oil phase in the gelled emulsion, preferably at least 10% wt, more especially at least 50% wt, particularly at least 80% wt. of that phase. They may be used as single compounds or as compound mixtures, e.g. plant or marine oils. The free fatty acids, the monoacyl glycerides and diacylglycerides may be prepared by full or partial hydrolysis of triacylglycerides, for example acid, base, or enzyme-catalysed hydrolysis, e.g. using lipases such as pancreatic lipases and/or lipases which may be produced from bacteria as fermentation products. Alkyl esters of essential fatty acids may be prepared by transesterification using the appropriate alkanol or by esterification of the free fatty acid with that alkanol. Where a free fatty acid is used, this may be in acid form or salt form (e.g. wholly or partially in salt form), and preferably constitutes 5 to 75% wt, especially 10 to 35% wt. of the essential fatty acid in the oil phase. Salt forms may be preferred.

The gelled oil-in-water emulsions herein described also find use as pharmaceuticals in the treatment or prevention of a range of medical conditions which are responsive to the chosen active agent(s). As will be appreciated, the nature of such conditions will be dependent on the selected active agent(s), but can readily be determined by those skilled in the art.

Any drug substance having a desirable therapeutic and/or prophylactic effect may be used. This includes drug substances which are lipophilic or hydrophilic.

Classes of suitable drug substances include, but are not limited to, any of the following: analgesics; anti-inflammatories; anti-cancer agents; cardiovascular agents; biological agents; anti-allergy agents (e.g. antihistamines); decongestants; anti-nausea agents, drugs affecting gastrointestinal function; drugs acting on the blood and blood-forming organs; drugs affecting renal and cardiovascular function;

anti-fungal agents; urological agents; hormones; antimicrobial agents, anti-epileptical agents; psycholeptical agents; antipsychotic agents; psychoanaleptical agents; anticholinesterase agents; and carotenoids.

Examples of specific drug substances which may find use in the compositions of the invention include: temazepam; diphenhydramine; zolpidem; triazolam; nitrazepam; testosterone; estradiol; progesterone; benzodiazepines; barbiturates; cyclosporine; insulin; calcitonin; dextromethorphan; pseudoephedrine; phenylpropanolamine; bromocryptine; apomorphine; selegiline; amitriptyline; dextroamphetamine; phentermine; mazindol; compazine; chlorpromazine;

perphenazine; fluoxetine, buspirone; clemastine; chlorpheniramine; dexochlorpheniramine; astemizole; loratadine; paracetamol; ketoprofen; naproxen; ibuprofen; sodium acetazolamide, acetyl salicylic acid, aminophylline, amiodarone hydrochloride, ascorbic acid, atenolol, bendroflumethiazide, calcium folinate, captopril, cetrizine hydrochloride, chloramphenicol sodium succinate, chlorpheniramine maleate, chlorpromazine hydrochloride, cimetidine hydrochloride, ciprofloxacin hydrochloride, clindamycin hydrochloride, clonidine hydrochloride, codeine phosphate, cyclizine hydrochloride, cyclophosphamide, sodium dexamethasone phosphate, sodium dicloxacillin, dicyclomide hydrochloride, diltiazem hydrochloride, diphenhydramine hydrochloride, disopyramide phosphate, doxepin hydrochloride, enalapril maleate, erythromycin ethylsuccinate, flecanide acetate, fluphenazine hydrochloride, folic acid, granisteron hydrochloride, guafenesin, haloperidol lactate, hydralazin hydrochloride, hydrochloroquine sulfate, hydromorphone hydrochloride, hydroxyzine hydrochloride, sodium indomethacin, isoniazid, isoprenaline hydrochloride, ketorolac trometamol, labetalol hydrochloride, lisinopril, lithium sulfate, mesoridazine benzylate, methadone hydrochloride, methylphenidate hydrochloride, methylprednisolone sodium succinate, metorprolol tartrate, metronidazole hydrochloride, metyldopa, mexiletine hydrochloride, molidone hydrochloride, morphine sulfate, naltrexone hydrochloride, neomycin sulfate, ondanstreon hydrochloride, orciprenaline sulfate, sodium oxacillin, oxybutynin chloride, oxycodone hydrochloride, paracetamol, penicillamine, pentoxifylline, petidine hydrochloride, sodium phenobarbital, potassium phenoxymethylpenicillin, phenylephrine hydrochloride, sodium phenytoin, potassium iodide, primaquine phosphate, procainamide hydrochloride, procarbazine hydrochloride, prochlorperazine maleate, promazine hydrochloride, promethazine hydrochloride, propranolol hydrochloride, pseudoephedrine hydrochloride, pyridostigmine bromide, pyridoxine hydrochloride, ranitidine hydrochloride, salbutamol sulfate, sodium ethacrynate, sotalol hydrochloride, sumatripan succinate, terbinafine hydrochloride, terbutaline sulfate, tetracycline hydrochloride, thioridazine hydrochloride, thiothixene hydrochloride, trifluoperazine hydrochloride, triprolidine hydrochloride, sodium valproate, vancomycin hydrochloride, vancomycin hydrochloride, verapamil hydrochloride, sodium warfarin, astaxanthin, lutein, CoQ10 and fenofibrate.

The quantity of drug substance per unit dose of the compositions of the invention will conveniently be in the range of 10 to 100% of the recommended daily dose for an adult or child.

Viewed from another aspect, the invention thus provides a gelled oil-in-water emulsion as herein described for use in therapy.

Viewed from still another aspect, the invention provides a gelled oil-in-water emulsion as herein described which contains at least one pharmaceutically active component for oral use in the treatment of a condition responsive to said pharmaceutically active component.

In another aspect the invention provides the use of a pharmaceutically active component in the manufacture of a medicament for oral use in the treatment of a condition responsive to said pharmaceutically active component, wherein said medicament is provided in the form of a gelled oil-in-water emulsion as herein described.

Corresponding methods of medical treatment form a further aspect of the invention. Viewed from a yet further aspect, the invention thus provides a method of treatment of a human or non-human animal subject (e.g. a patient) to combat a condition responsive to a pharmaceutically active agent, said method comprising the step of orally administering to said subject a pharmaceutically effective amount of said agent in the form of a gelled oil-in-water emulsion as herein described.

In another aspect the invention provides the use of a gelled oil-in-water emulsion as herein described as a nutraceutical. Corresponding methods of administering the gelled oil-in-water emulsion in order to achieve a nutraceutical effect also form part of the invention.

Viewed from another aspect the invention thus provides a method of administering an active agent to a human or non-human animal subject to enhance and/or maintain said subject's health or wellbeing, said method comprising the step of orally administering to said subject a nutraceutically effective amount of an active agent in the form of a gelled oil-in-water emulsion as herein described.

In another aspect the invention provides the use of a gelled oil-in-water emulsion as herein described as a nutraceutical.

When used in any of the above treatments or methods, or as nutraceutical supplements or pharmaceutical formulations, an effective amount of the active agent can readily be determined.

The effective dose level for any particular subject will depend on a variety of factors including the disorder and its severity, the identity and activity of the particular composition, the age, bodyweight, general health of the subject (e.g. patient), timing of administration, duration of treatment, other drugs being used in combination with the treatment, etc. It is well within the skill of those in the art to select the desired dose to achieve the desired therapeutic effect.

The invention will now be described further with reference to the following non-limiting Examples and the accompanying figures in which:

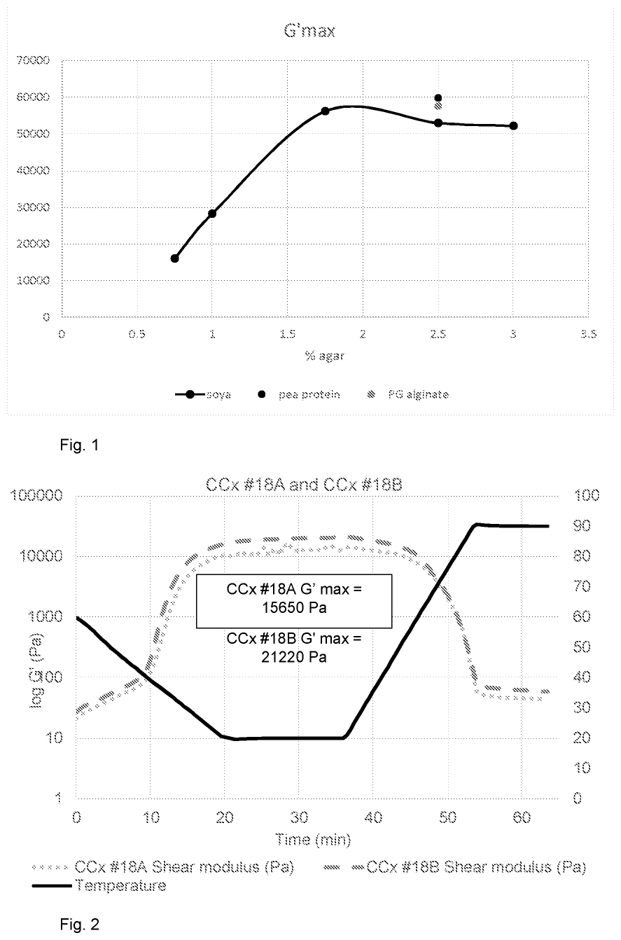

FIG. 1 shows the dynamic storage modulus (G′ max) for agar-based gelled oil-in-water emulsions containing different surfactants.

FIG. 2 shows the dynamic storage modulus (G′ max) for gelled oil-in-water emulsions according to the invention.

FIG. 3 shows the hardness (force) of gelled oil-in-water emulsions according to the invention measured in accordance with a texture profile analysis (TPA) test.

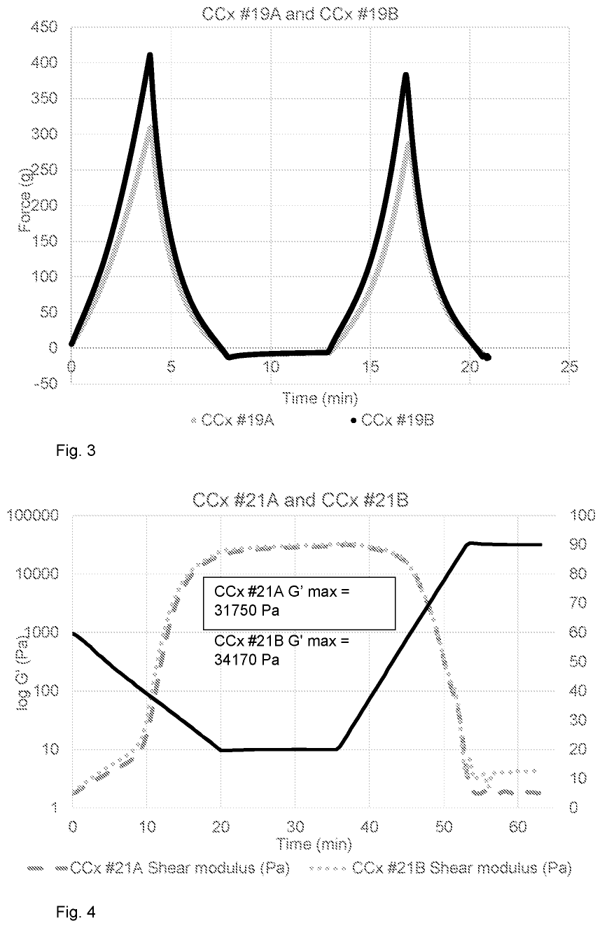

FIG. 4 shows the dynamic storage modulus (G′ max) for gelled oil-in-water emulsions according to the invention.

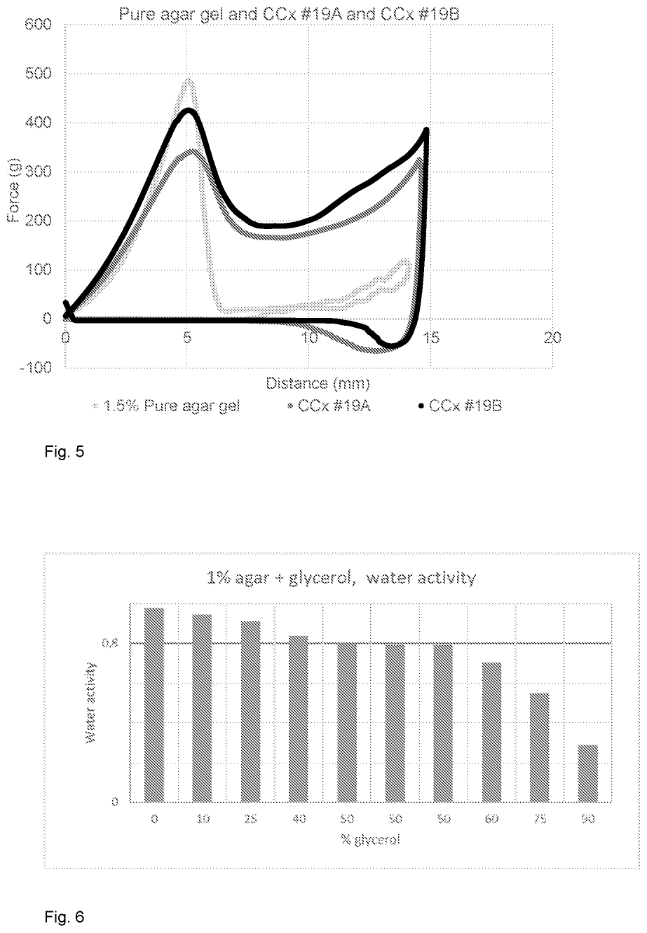

FIG. 5 shows the hardness (force) of gelled oil-in-water emulsions according to the invention when subjected to large scale deformation.

FIG. 6 shows the measured water activity of aqueous agar gels with increasing glycerol content.

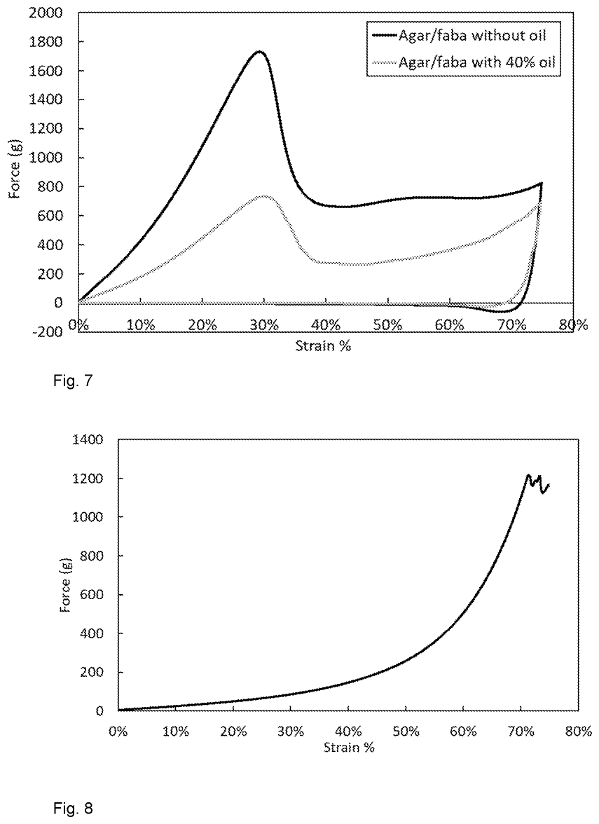

FIG. 7 shows the hardness (force) of gelled oil-in-water emulsions according to the invention compared to a pure agar gel.

FIG. 8 shows the hardness (force) of gelled oil-in-water emulsions containing gelatin as the gelling agent.

### EXAMPLES

### Test Methods:

### 1. Rheological Characterisation of Agar and Agar Emulsion Gels

### 1a —Small Scale Deformation

Rheological analyses on the gels were performed with a rheometer (Malvern Kinexus ultra+, Westborough, United States). The lower plate was KNX0127, 50 mm diameter curved sandblasted lower plate. The upper geometry was CP4/40 40 mm diameter 4° angle cone for gelatin emulsion gels and serrated PP40X SW1648 SS for agar gels and agar emulsion gels. Instrument calibration (zero gap) was performed prior to analysis. After gel preparation, approximately 2 grams of gel was placed on the lower plate, which was heated up to 60° C. The rheometer was operated in 0.1% shear strain controlled mode and the frequency was set to 1 Hz. The chosen strain was confirmed to be within the linear viscoelastic region for all samples. In order to avoid evaporation, the gelatin emulsion gel samples were covered with silicone oil (10 cS fluid, Dow Corning, UK) prior to measurement. The viscoelastic properties of the sample were obtained by using a temperature gradient of 2° C./min, with a start and end temperature at 60° C. and a holding time of 15 min at 20° C. for the gelatin emulsion gels. For agar emulsion gels, the end temperature was 90° C. and oscillation continued for 10 minutes at 90° C. The results were analyzed using rSpace for Kinexus software. The gelling and melting temperatures of the samples were estimated as the temperature at which the phase angle corresponded to 45° in the cooling and heating process, respectively. The maximum storage modulus (G′) (Pa) was determined as the highest measurement point during curing at 20° C.

### 1b —Large Scale Deformation

Texture properties of the gels were analysed with TA.XT plusC Texture Analyser (Stable Micro Systems Ltd., UK). Upon preparation, the gels were cast using cylindrical molds of standard dimensions (19.6 mm height, 8 mm diameter). The gels were cured at ambient temperature for 18 hours prior to analysis. Single compression analysis and the standard texture profile analysis (TPA) were performed using a 5 kg load cell. A P/35 35 mm diameter cylinder aluminum probe supplied by Stable Micro Systems Ltd. was used. For the 75% large and strain single compression, pre-test and post-test speeds were 2 mm/sec, while the test speed was 0.5 mm/sec and the trigger force was 5 grams. Strain height was measured automatically during compression. Max stress (g) and strain at failure (%) data was obtained from the fraction moment of the gels. Gradient (N/m) was calculated by the ratio of force at 2% and 3% strain. Young's modulus (N/m²) was calculated from gradient by following equation:

$$Young ' s modulus ( N / {m}^{2} ) = \frac{Gradient ( N / m ) \times height of the gel ( m )}{Area of the gel ( {m}^{2} )}$$

Area of the gel is the contact area of the gel with the probe.

2. Texture profile analysis (TPA test)

The standard TPA was carried out at 20% strain double compression at room temperature applying a TA.XT plusC Texture Analyser (Stable Micro Systems Ltd., UK) using a 5 kg load cell and a P/35 aluminum probe. Cylindrical molds of standard dimensions (19.6 mm height, 8 mm diameter) were used. The gels were cured at ambient temperature for 18 hours prior to analysis. Pre-test, test and post-test speeds were 1 mm/sec and the trigger force was 5 grams. Strain height was measured automatically during compression. Hardness, adhesives, resilience, cohesion, springiness, gumminess and chewiness parameters were measured. The data were analyzed with the Exponent connect software.

3 Syneresis measurements

Syneresis measurements were based on weight loss of the gels. The gel was weighed and sealed with an air- and moisture-tight aluminum foil. Upon freezing at −20° C. and thawing at ambient temperature, and after removing excess liquid, the gel was weighed again and the difference in gel weight was normalized to percentage loss.

4. Water activity measurements

Water activity was measured with HygroPalm HC2-AW (Rotronic, Switzerland) at ambient temperature. The sample was placed into the measurement chamber and the water activity was recorded after 45 minutes.

### Example 1—Gelled oil-in-water compositions and preparation method

### Composition:

Typical gelled oil-in-water compositions according to the invention are listed in the following table. It will be understood that any component which may be present in an amount of 0 wt. % is optional.

| Component | Wt. % |
|---|---|
| Agar (gelling agent) | 0.5-2.5 |
| Plant-based surfactant(s) | 0.25-3.5 |
| Bulking agent(s), e.g. sugar alcohol(s) | 30-60 |
| pH modifier(s) | 0-6 |
| Oil(s) | 10-50 |
| Viscosity modifier(s) | 0-5 |
| Plasticiser(s), e.g. glycerol | 0-60 |
| Anti-foaming agent(s) | 0-0.5 |
| Anti-Oxidant(s) | 0-3 |
| Sweetening agent(s) | 0-3 |
| Flavouring agent(s) | 0.05-3 |
| Colouring agent(s) | 0.001-3 |
| Pharmaceutical agent(s) | 0-10 |
| Nutraceutical agent(s) | 0-10 |
| Water | to 100 |

Method of preparation:

In the following method, the pH modifier is an organic acid/base buffer system consisting of trisodium citrate and malic acid, and the plasticiser (when present) is glycerol.
1. Mix agar, sugar alcohols and any sweetening agent(s) into a homogeneous powder mixture.
2. Weigh sterile water into a bottle and add the powder mixture to the water.
3. Place bottle in a water bath at 90° C. and mix with magnetic stirring for 30 minutes at 100 rpm. If glycerol is used, heat glycerol separately for 30 minutes at 60° C.
4. Reduce the temperature to 60° C. and mix the water phase with magnetic stirring for an additional 30 minutes (approx. 60 minutes in total). If glycerol is used, add the heated glycerol to the mixture with a syringe.
5. In a beaker, mix the oil together with any flavouring and/or colouring agent(s) and pre-heat to 50° ° C. for 30 minutes (at minute 40 of total 60 minutes).
6. When the ingredients are completely dissolved, slowly add the surfactant and trisodium citrate to the water phase. Where the surfactant contains any plant protein, it is added at a temperature below the denaturation temperature of the plant protein. Mix for 10 minutes and slowly add malic acid (carefully and gradually). Mix the mass for 10 more minutes.
7. If using an anti-foaming agent, add half of this agent and leave for 1 minute without stirring.
8. Weigh the bottle containing the water phase and vacuum the mass. Add the lost water (heated) and mix for 1 minute.
9. Add the oil phase into the agar mass (water phase) and homogenise the two phases for approx. 10 minutes using a high speed blender, such as an Ultra-Turrax.
10. If appropriate, add the second half of the (heated) anti-foaming agent and leave for 1 minute without stirring.
11. Weigh the bottle and vacuum the mass. Add the lost water (heated) and mix for 1 minute.
12. If desired, fill the resulting emulsion into blisters of a blister pack and seal.

Example 2 - Gelled oil-in-water emulsion - typical formulation

| Component | Wt. % |
|---|---|
| Agar (gelling agent) | 0.5-2.5 |
| Plant-based surfactant | 0.25-3.5 |
| Xylitol (bulking agent) | 20-40 |
| Sorbitol (bulking agent) | 10-25 |
| Malic acid (pH modifier) | 0-2 |
| Trisodium citrate (pH modifier) | 0-4 |
| Oil | 10-50 |
| Gum Arabic (surfactant) | 0-2 |
| Locust Bean Gum (viscosity modifier) | 0-3 |
| Glycerol (plasticiser) | 0-60 |
| Anti-foaming agent(s) | 0-0.5 |
| Sweetening agent(s) | 0-3 |
| Flavouring agent(s) | 0.05-3 |
| Colouring agent(s) | 0.001-3 |
| Anti-oxidant(s) | 0-3 |
| Water | to 100 |

The emulsion can be prepared according to the general method in Example 1.

Example 3 - Gelled oil-in-water emulsion containing algae oil

| Ingredient | Wt. % |
|---|---|
| Agar HDR¹ | 1.65 |
| Soy protein isolate² | 1.40 |
| Sorbitol | 14.36 |
| Xylitol | 29.02 |
| Ascorbic acid | 0.45 |
| Malic acid | 0.30 |
| Trisodium citrate | 0.75 |
| Algae oil | 25.00 |
| Lemon Lime flavor | 1.20 |
| Paprika extract | 0.08 |
| Witafrol 7420 (AF) | 0.08 |
| Stevia | 0.10 |
| Water | 25.70 |

¹Gelagar HDR 800 (B.V. srl, Italy)
²Supro 590 (PHH)

The emulsion is prepared according to the general method in Example 1.

Example 4 - Gelled oil-in-water emulsion containing sunflower oil

| Ingredient | Wt. % |
|---|---|
| Agar HDR¹ | 1.75 |
| Soy protein isolate² | 1.50 |
| Sorbitol | 16.00 |
| Xylitol | 31.43 |
| Ascorbic acid | 0.45 |
| Malic acid | 0.40 |
| Trisodium citrate | 0.90 |
| Sunflower Oil | 20.00 |
| Lemon Lime flavor | 1.20 |
| Paprika extract | 0.08 |
| Witafrol 7420 (AF) | 0.08 |
| Stevia | 0.10 |
| Water | 26.20 |

¹Gelagar HDR 800 (B.V. srl, Italy)
²Supro 590 (PHH)

The emulsion is prepared according to the general method in Example 1.

Example 5 - Gelled oil-in-water emulsion containing algae oil

| Ingredient | Wt. % |
|---|---|
| Agar | 1.50 |
| Faba bean protein | 1.50 |
| Sorbitol | 14.50 |
| Xylitol | 29 |
| Malic acid | 1 |
| Trisodium citrate | 2 |
| Algae oil | 25 |
| Flavouring/colouring/sweetening | 1.50 |
| agents/anti-foaming agent/antioxidant | |
| Water | 24 |

The emulsion is prepared according to the general method in Example 1.

Example 6 - Gelled oil-in-water emulsion containing algae oil

| Ingredient | Wt. % |
|---|---|
| Agar | 1.50 |
| Faba bean protein | 1.50 |
| Trehalose | 21 |
| Sucrose | 21 |
| Algae oil | 25 |
| Flavouring/colouring/sweetening | 1.50 |
| agents/anti-foaming agent/antioxidant | |
| Water and citric acid to pH 4.5 | 28.5 |

The emulsion is prepared according to the general method in Example 1 in which trehalose and sucrose are employed as the sugar alcohols. Following homogenisation of the two phases, 50 wt. % citric acid is added until the pH reaches 4.5.

Example 7 - Multi-vitamin supplement

| Ingredient | Wt. % |
|---|---|
| Agar | 1.50 |
| Faba bean protein | 1.50 |
| Sorbitol | 14.5 |
| Xylitol | 29 |
| Malic acid | 1 |
| Sodium tricitrate | 2 |
| Vegetable oil¹ | 25 |
| Flavouring/colouring/sweetening | 1.50 |
| anti-foaming agent/antioxidant | |
| Water² | 24 |

¹Neutral carrier oil containing 10 mcg Vitamin D and 45 mcg Vitamin K
²Water phase containing 2 mcg Vitamin B12

The emulsion is prepared according to the general method in Example 1. Vitamin D and Vitamin K are added to the oil in Step 5 and Vitamin B12 is added to the water phase in Step 6.

Example 8 - Multi-mineral supplement

| Ingredient | Wt. % |
|---|---|
| Agar | 1.50 |
| Faba bean protein | 1.50 |
| Sorbitol | 18.5 |
| Xylitol | 31 |
| Malic acid | 1 |
| Sodium tricitrate | 2 |
| MCT oil | 10 |
| Flavouring/colouring/sweetening | 1.50 |
| agents/anti-foaming agent/antioxidant | |
| Water¹ | 33 |

¹Water phase containing 150 mcg iodine, 40 mcg selenium, 20 mg iron and 2.5 mg zinc

The emulsion is prepared according to the general method in Example 1. The minerals are added to the water phase in Step 6.

Example 9 - Calcium supplement

| Ingredient | Wt. % |
|---|---|
| Agar | 1.50 |
| Faba bean protein | 1.50 |
| Sorbitol | 14.50 |
| Xylitol | 29 |
| Calcium Phosphate Dibasic - CaHPO₄ | 20 |
| Malic acid | 1 |
| Sodium tricitrate | 2 |
| Vegetable oil¹ | 4 |
| Flavouring/colouring/sweetening | 1.50 |
| agents/anti-foaming agent/antioxidant | |
| Water | 25 |

¹Vegetable oil containing 400 IU Vitamin D3

The emulsion is prepared according to the general method in Example 1. The calcium phosphate is added to the water phase together with the faba bean protein and sodium tricitrate.

Example 10 - Multivitamin supplement

| | | Amount of | |
|---|---|---|---|
| Ingredient | Wt. % | Active | |
| Water | 26.00 | | |
| Agar HDR | 1.75 | | |
| Soy protein isolate | 1.50 | | |
| Sorbitol | 15.00 | | |
| Xylitol | 31.06 | | |
| Trisodium citrate dihydrate | 0.90 | | |
| Sunflower oil | 20.00 | | |
| Lemon Lime flavour | 1.20 | | |
| Paprika extract | 0.075 | | |
| Witrafrol 7420 (AF) | 0.075 | | |
| Stevia | 0.10 | | |
| Vitamin C (as Ascorbic Acid) | 1.18 | 15 | mg |
| Vitamin B3 (as Nicotinamide) | 0.56 | 8 | mg |
| Vitamin E (as D-α-Tocopherol) | 0.54 | 5 | mg |
| Vitamin B6 (as Pyridoxine HCl) | 0.064 | 0.7 | mg |
| Vitamin A (as Retinyl palmitate) | 0.029 | 200 | mcg |
| Vitamin D3 (as Cholecaliferol) | 0.016 | 5 | mcg |
| Iodine (as Potassium Iodide) | 0.011 | 30 | mcg |
| Vitamin B12 (as Cyanocobalamin) | 0.010 | 1.25 | mcg |
| Folic acid | 0.0087 | 100 | mcg |
| D-biotin | 0.0020 | 25 | mcg |

The emulsion is prepared according to the general method in Example 1. The fat soluble vitamins (E, A, D3) are mixed into the oil as in Example 7, and the water soluble vitamins (C, B3, B6, B12, folic acid, D-biotin) as well as iodine are mixed in as the CaHPO₄ is mixed in Example 8.

Example 11 - Gelled oil-in-water emulsion containing corn oil

| Ingredient | Wt. % |
|---|---|
| Water | 33.0 |
| Locust Bean Gum | 0.20 |
| Agar HDR | 1.80 |
| Soy protein isolate | 0.90 |
| Glycerol | 34.72 |
| Malic acid | 1.10 |
| Trisodium citrate dihydrate | 2.20 |
| Corn oil | 25.00 |
| Lemon flavour | 0.90 |
| Paprika extract | 0.075 |
| Witrafrol 7420 (AF) | 0.075 |
| Stevia | 0.10 |

The emulsion is prepared according to the general method in Example 1.

Example 12 - Gelled oil-in-water emulsion containing corn oil

| Ingredient | Wt. % |
|---|---|
| Water | 12.65 |
| Agar HDR | 2.00 |
| Soy protein isolate | 0.90 |
| Glycerol | 55.00 |
| Malic acid | 1.10 |
| Trisodium citrate dihydrate | 2.20 |
| Corn oil | 25.00 |
| Lemon flavour | 0.90 |
| Paprika extract | 0.075 |
| Witrafrol 7420 (AF) | 0.075 |
| Stevia | 0.10 |

The emulsion is prepared according to the general method in Example 1.

### Example 13—Packaging

Blister packs:

Prior to setting, the emulsions produced in any of Examples 1-12 may be filled into blister trays made from a metal/plastic laminate or a plastic film over which a plastic/metal foil laminate is heat sealed. Blister trays with pre-formed cavities may be formed from laminated materials such as Tekniflex® Aclar® VA10600 (TekniPlex), Perlalux® (Perlen Packaging), Formpack® (Amcor), and Regula® (Constantia Flexibles).

A liquid emulsion produced in any of Examples 1-12 is filled into blister trays using a syringe and ensuring that the cavities are filled evenly and fully. The blister trays are then flushed with nitrogen for 5-10 seconds, and sealed with a metal/plastic or metal/heat-seal lacquer cover foil by applying a flat iron set at 160° C. for 2-4 seconds. The samples are left to cure for 24 hours at room temperature, and submitted to a controlled holding chamber at 40° C. for 30 days, 65% RH. On day 5, 10, 15, 20, 25 and 30, samples were withdrawn from the controlled chamber. After 24 hours at room temperature, the blister packs are opened. The amount of residues adhering to the trays and the force required to remove the unit dose from the packs is noted on a scale from 1-9, where 1 indicates no adhesion and very little force required to remove the unit dose (“popping out”), and 9 is full adhesion to the foil and the unit dose needs to be torn from the foil. Each of the laminated materials listed above gives scores of 1, 2 or 3 (mainly 1 or 2) in each test.

Strips:

Prior to setting, the emulsions produced in any of Examples 1-12 may be extruded into individual strips which, once set, are then sealed into individual plastic/metal foil laminate sachets. Alternatively, a single extruded strip, once set, may be cut into individual strips according to need prior to packaging.

### Example 14—Coated Gelled Emulsions

The set emulsions produced in any of Examples 1-12 may be coated with a sorbitol solution comprising sorbitol (80 wt. %), lemon flavour (0.15 wt. %), yellow colour (0.5 wt. %) and water (ad 100 wt. %). The coating solution may be cured at 99-95° C. for 4-5 hours before application. Coating is carried out by dipping or panning at 20-45° C. Several layers of coating material may be added with drying between each layer until the final composite layer is hard.

Alternatively, prior to setting, the liquid emulsion prepared in any of Examples 1-12 may be filled into soft capsule shells. The capsule shell material may typically be a sugar, e.g. sucrose, fructose, maltose, xylitol, maltitol or sorbitol, but may additionally contain hydrocolloid materials such as carrageenan, alginate, pectin, cellulose, modified cellulose, starch, modified starch or gum arabic. The capsule shell may contain further ingredients such as artificial sweeteners, colours, flavours and anti-oxidants.

### Example 15—Effect of Different Surfactants on Dynamic Storage Modulus (G′ Max)

Gelled oil-in-water emulsions containing 2.5 wt. % agar were prepared using soy bean protein, pea protein (Nutralys F85M), propylene glycol alginate (degree of esterification: 84%) (PG alginate), Tween 80 and LACTEM as surfactants. Gelled oil-in-water emulsions having agar concentrations ranging from 0.75 to 3 wt. % were also produced using soy bean protein. All formulations were tested for their rheology characteristics.

The following formulation containing 2.5 wt. % agar as gelling agent was prepared according to the general protocol in Example 1:

| Ingredients | Wt. % | Wt. (in 50 g) |
|---|---|---|
| Water | 24.36 | 12.18 |
| Agar HDR | 2.50 | 1.25 |
| Xylitol | 29.00 | 14.5 |
| Sorbitol | 14.95 | 7.475 |
| Surfactant¹ | 0.75 | 0.375 |
| Malic acid | 1.16 | 0.58 |
| Trisodium citrate dihydrate | 2.28 | 1.14 |
| Corn oil | 25.00 | 12.5 |
| Total | 100.00 | 50 |

¹Soy bean protein, pea protein, PG alginate, Tween 80 or LACTEM

Formulations with agar concentrations in the range 0.75 to 3.0 wt. % were also prepared using soy bean protein as a surfactant. Any change in agar concentration was compensated by an equivalent change in sorbitol content.

Soy bean protein, pea protein and PG alginate are high molecular weight surfactants (i.e. “macromolecular”), whereas Tween 80 and LACTEM are low molecular weight surfactants. Tween 80 is a polysorbate surfactant derived from polyethoxylated sorbitan and oleic acid. LACTEM consists of lactic acid esters of mono and diglycerides.

Stable emulsions could not be prepared with Tween 80 or LACTEM. In respect of all other formulations, the dynamic storage modulus (G′ max) was measured according to the small scale deformation test described herein. The results are shown in FIG. 1. With increasing agar concentration it could be observed that the small scale deformation modulus of the solid emulsion increased when using the macromolecular surfactants (soy/pea protein and PG alginate). Although not wishing to be bound by theory, this is believed to be due to a friction layer around the droplets created by an uneven distribution of large molecules (“hairy”), which provides a “semi-active filler” effect. This effect increases with increasing agar concentration. All macromolecular surfactants gave stable emulsions.

Example 16 - Press-testing
The following formulation containing 2.5 wt. % agar as gelling
agent was prepared according to the general protocol in Example 1:

| Ingredient | Wt. % | Wt. (in 50 g) |
|---|---|---|
| Water | 24.36 | 12.18 |
| Agar HDR | 2.50 | 1.25 |
| Xylitol | 29.00 | 14.5 |
| Sorbitol | 14.95 | 7.475 |
| Soy protein | 0.75 | 0.375 |
| Malic acid | 1.16 | 0.58 |
| Trisodium citrate dihydrate | 2.28 | 1.14 |
| Corn oil | 25.00 | 12.5 |
| Total | 100.00 | 50 |

Formulations with agar concentrations in the range 0.25 to 3.0 wt. % were also prepared. The change in agar concentration was compensated by an equivalent change in sorbitol content.

Whilst still liquid, the emulsions (1 ml) were poured into standard non-stick blister foil packs and sealed with a flat iron at 150° C. After 24 hours at room temperature the agar emulsions had solidified and press-testing was carried out to evaluate the minimum agar concentration at which the gelled tablets could be squeezed out of the blister forms without breaking. The lowest agar concentration that could withstand this without breaking into pieces was 0.75 wt. %. This corresponds to a G′ max of around 15 kPa. The highest acceptable agar concentration was found to be 2.5 wt. % based on visual observations where the solution before setting became very thick. That corresponded to a measured viscosity at 55° C. of 60 Pa·s at a shear rate of 1/s and 23 Pas at a shear rate of 10/s.

### Example 17—Effect of Different Surfactants

Tests were carried out to assess the impact of the molecular weight of the surfactant. As reported in Example 15, the high molecular weight surfactants—soy and pea proteins and propylene glycol alginate (PG alginate)—provided a stable emulsion having a high dynamic storage modulus (G′ max). This was attributed to formation of a ‘hairy’ droplet surface formation acting as a semi-active filler through increased friction. In the following series of experiments, hydroxy propyl methyl cellulose (HPMC) materials having an identical degree of substitution but with varying weight average molecular weights were tested. HPMC materials were obtained from Shin-Etsu Tylose GmbH having the following properties:

| | Weight Average |
|---|---|
| | Molecular Weight, Mw |
| HPMC Material | (kDa)¹ |
| Metolose SB-4 | 24 |
| Metolose 90SH-100SR | 94 |
| Metolose 90SH-15000SR | 435 |

¹Weight average molecular weight was determined based on a conversion factor from viscosity to Mw provided by the manufacturer (Mw = 40000 x log η + 880 × (log η)⁴ wherein Mw = weight-average molecular weight; η = solution viscosity).

The basic formulation used in this experimental series was as follows:

| | | Wt. |
|---|---|---|
| Ingredient | Wt. % | (in 50 g) |
| Water | 24.36 | 12.18 |
| Agar¹ | 1.00 | 0.5 |
| Xylitol | 29.00 | 14.5 |
| Sorbitol | 16.45 | 8.225 |
| Surfactant | 0.75 | 0.375 |
| Malic acid | 1.16 | 0.58 |
| Trisodium citrate dihydrate | 2.28 | 1.14 |
| Corn oil | 25.00 | 12.5 |
| Total | 100.00 | 50 |

¹B&V Gelagar HDR 800 (B.V. srl, Italy)

Preparation of oil-in-water emulsions:
1. Agar and sugar alcohols were mixed as a dry powder and added to a bottle with the correct amount of water. The bottle was put in a water bath at 90° C. with magnetic stirring for 30 minutes.
2. The bottle was transferred to a water bath at 50° C., equilibrated for 15 minutes. This was the stage where HPMC surfactant was added, and these mixtures were left for 30 minutes to allow the HPMC to dissolve.
3. The pre-heated (50° C.) corn oil was added and homogenisation was carried out using an Ultra-Turrax for 5 minutes.
4. The resulting emulsion was put back at 55° C. for 10 minutes before rheological examination.
5. Rheological experiments were carried out applying a Kinexus Ultra+Rheometer equipped with a C 4/40 measuring geometry. Strain 0.1%, a 1 Hz frequency, a temperature gradient from 50 to 20° C. and a holding time of 20 minutes at 20° ° C. was applied before a G′ value for the different systems was recorded.

The results are presented in Table 1:

TABLE 1

| | Average Mw | G′ max |
|---|---|---|
| Surfactant | (kDa) | (kPa) |
| Metolose SB-4 | 24 | 9.9 |
| Metolose 90SH-100SR | 94 | 20.2 |
| Metolose 90SH-15000SR | 435 | 19.5 |

An increase in weight average molecular weight from 24 to 94 kDa led to a doubling of the G′ max value when using HPMC as a macromolecular surfactant. No further increase with molecular weight was observed beyond that. It was observed by visual inspection that the lowest Mw HPMC surfactant gave higher syneresis (release of water phase) than the higher Mw ones. All HPMC formulations were able to retain the oil when exposed to mechanical stress. Higher Mw surfactants thus give additional benefits other than increased gel strength, i.e. less syneresis and better retention of oil.

### Example 18—Tests on Gelled Oil-in-water Compositions Containing Agar and Gum Arabic to Show the Effect of Increasing Agar Concentration

50 g formulations containing the following ingredients were prepared using methodology analogous to that in Example 1.

| | Example | Example |
|---|---|---|
| | 18A | 18B |
| Ingredients | Wt. % | Wt. % |
| Agar¹ | 1.13 | 1.50 |
| Gum Arabic | 1.00 | 1.00 |
| Water | 24.88 | 24.5 |
| Xylitol | 29 | 29 |
| Sorbitol | 14.5 | 14.5 |
| Sodium tricitrate | 2 | 2 |
| Malic acid | 1 | 1 |
| Corn oil | 25 | 25 |
| Faba bean protein² | 1.5 | 1.5 |
| Total | 100.0 | 100.0 |

¹Gelagar HDR 800 (B.V. srl, Italy)
²Vestkorn Faba Protein F65X (Vestkorn A/S, Denmark)

Droplet sizes and size distributions were measured using a Malvern Mastersizer 3000 (Worcestershire, UK) connected to a Hydro MV, wet dispersion unit (Malvern, Worcestershire, UK). Analysis of the data was performed using the manufacturer's software (Mastersizer 3000, v1.0.1). Testing was carried out by dissolving and diluting the gelled emulsion in a 10% (v/v) HCI solution (1:100) at 50° C. The refractive index of water and corn oil was set to 1.33 (solvent) and 1.47 (dispersed phase), respectively, and the absorption index of the dispersed droplets set to 0.01. To avoid multiple scattering or low intensity of the scattered light, each dissolved emulsion was added to the dispersion unit (containing ˜125 ml water), until an obscuration of approximately 10% was obtained. Droplet size distributions for the different emulsions are shown in Table 2.

TABLE 2

| | D [4;3] | D [3;2] | Dx(10) | Dx (50) | Dx (90) |
|---|---|---|---|---|---|
| Example 18A | 8.75 | 3.59 | 1.66 | 7.56 | 16.98 |
| Example 18B | 13.64 | 4.756 | 2.908 | 12.4 | 25.64 |

Dynamic storage modulus (G′ max) was measured according to the small scale deformation test described herein. The results in FIG. 2 show the shear modulus (elastic component) of the resulting emulsion as a function of temperature and time. G′ max for the formulation containing 1.13 wt. % agar (Example 18A) was 15,650 Pa, whereas that for the formulation containing 1.50 wt. % agar (Example 18B) was 21,220 Pa. This confirms an increase in the strength of the gel with increasing agar concentration. Both formulations exhibited a ‘solid-like’ nature over a wide temperature range.

### Example 19—Tests on Gelled oil-in-water Compositions Containing Agar with and without Gum Arabic

50 g formulations containing the following ingredients were prepared using methodology analogous to that in Example 1.

| | Example | Example |
|---|---|---|
| | 19A | 19B |
| Ingredients | Wt. % | Wt. % |
| Agar¹ | 1.50 | 1.50 |
| Gum Arabic | 1.00 | 0 |
| Water | 24.5 | 24.5 |
| Xylitol | 29 | 29.5 |
| Sorbitol | 14.5 | 15 |
| Sodium tricitrate | 2 | 2 |
| Malic acid | 1 | 1 |
| Corn oil | 25 | 25 |
| Faba bean protein² | 1.5 | 1.5 |
| Total | 100.0 | 100.0 |

¹Gelagar HDR 800 (B.V. srl, Italy)
²Supplied by Vestkorn

Droplet sizes and size distributions were measured as described in Example 18. The results are shown in Table 3.

TABLE 3

| | D [4;3] | D [3;2] | Dx (10) | Dx (50) | Dx (90) |
|---|---|---|---|---|---|
| Example 19A | 14.76 | 5.248 | 3.196 | 13.16 | 28.14 |
| Example 19B | 24.12 | 7.55 | 6.248 | 22.3 | 44.18 |

Texture analysis was carried out using a standard TPA test as described herein. The results are shown in Table 4 and in FIG. 3.

TABLE 4

| | Ex. 19A | Ex. 19B |
|---|---|---|
| | (7 samples) | (10 samples) |
| Hardness (g) | 312.9 ± 8.9 | 413.5 ± 7.7 |
| Adhesiveness (g · sec) | −45.5 ± 11.7 | −42.6 ± 9.8 |
| Resilence (%) | 59.6 ± 1.5 | 62.2 ± 0.5 |
| Cohesion | 0.8 ± 0.0 | 0.8 ± 0.0 |
| Springiness (%) | 95.4 ± 1.1 | 96.9 ± 0.8 |
| Gumminess | 257.8 ± 7.4 | 349.8 ± 7.9 |
| Chewiness | 245.8 ± 6.6 | 338.8 ± 9.2 |

In general, the presence of gum arabic was found to result in gelled emulsions having smaller droplet sizes and which are slightly softer.

### Example 20—Droplet Size and Size Distribution

50 g formulations containing the following ingredients were prepared using methodology analogous to that in Example 1.

| | Example | | |
|---|---|---|---|
| | 20 | Temperature | Mixing time |
| | Wt. % | (° C.) | (min) |
| Agar | 1.5 | 90 | 30 |
| Xylitol | 29 | | |
| Sorbitol | 14.5 | | |
| Stevia | 0.1 | | |
| Water | 24.125 | | |
| Malic acid | 1 | 65 | 10 |
| Faba bean protein | 1.5 | | 45 |
| (Vestkorn) | | | |
| Trisodium Citrate | 2 | | 10 |
| Paprika extract | 0.075 | 50 | 2 |
| Lemon flavor | 1.2 | | |
| Algae oil | 25 | | |
| Total | 100 | | Homogenization |
| | | | 7 min at speed |
| | | | 5 |

Droplet sizes and size distributions were measured as described in Example 18. Rheological analysis was carried out according to the small scale deformation test described herein. In accordance with standard rheological measurement methods, Tg and Tm were determined when the phase angle dropped below or went above 45° under the given temperature gradient, strain and frequency. The results are provided in Table 5 and in FIG. 4.

TABLE 5

| | D | D | Dx | Dx | Dx | G′ max | Tg | Tm |
|---|---|---|---|---|---|---|---|---|
| | [4; 3] | [3; 2] | (10) | (50) | (90) | (kPa) | (° C.) | (° C.) |
| Ex. 20 | 22.08 | 7.976 | 8.32 | 20.76 | 38.34 | 15 | 39.5 | 88.7 |

### Example 21—Droplet Size and Size Distribution

50 g formulations containing the following ingredients were prepared using methodology analogous to that in Example 1.

| | Example | Example | | |
|---|---|---|---|---|
| | 21A | 21B | Temperature | Mixing time |
| | Wt. % | Wt. % | (° C.) | (min) |
| Agar | 1.75 | 2 | 90 | 30 |
| Xylitol | 28.875 | 28.75 | | |
| Sorbitol | 14.375 | 14.25 | | |
| Stevia | 0.1 | 0.1 | | |
| Water | 24.125 | 24.125 | | |
| Malic acid | 1 | 1 | 65 | 10 |
| Faba bean protein | 1.5 | 1.5 | | 45 |
| (Vestkorn) | | | | |
| Trisodium citrate | 2 | 2 | | 10 |
| Paprika extract | 0.075 | 0.075 | 50 | 2 |
| Lemon flavor | 1.2 | 1.2 | | |
| Algae oil | 25 | 25 | | |
| Total | 100 | 100 | | Homogenization |
| | | | | 7 min at speed |
| | | | | 5 |

Droplet sizes and size distributions were measured as described in Example 18. The results are provided in Table 6.

TABLE 6

| | D [4;3] | D [3;2] | Dx (10) | Dx (50) | Dx (90) |
|---|---|---|---|---|---|
| Example 21A | 23.88 | 7.81 | 5.224 | 22.7 | 43 |
| Example 21B | 15.34 | 7.21 | 7.862 | 14.98 | 24.14 |

### Example 22—Large Scale Deformation of Gelled oil-in-water Emulsion

Experiments were carried out to compare the large scale deformation of agar-based emulsions prepared according to Examples 19A and 19B and that of pure aqueous agar gels. Pure aqueous agar gels were prepared by mixing agar (2 wt. %) and Milli-Q-water (MQ-H₂O) at 90° C. The mixture was cooled down to ambient temperature for further characterization of the gels.

Tests were carried out according to the large scale deformation method described herein. The results are shown in FIG. 5 and in Table 7.

TABLE 7

| | Young's | | |
|---|---|---|---|
| | modulus | Max stress | Strain at failure |
| | (N/m²) | (g) | (%) |
| 1.5% pure agar gel | 29354.2 ± 2444.8 | 495.2 ± 42.1 | 26.8 ± 0.9 |
| (10 samples) | | | |
| Example 19A | 48017.8 ± 1749.5 | 340.4 ± 17.4 | 25.7 ± 1.2 |
| (6 samples) | | | |
| Example 19B | 64100.9 ± 5258.2 | 423.4 ± 25.5 | 25.1 ± 1.2 |
| (6 samples) | | | |

The Young's modulus (or initial slope of the force/deformation curve in this context) is somewhat higher for the agar-based gelled emulsions according to the invention which means these provide more resistance at very low deformation. However, these compositions conserved much more structure after failure (at around 25% strain) compared to the pure aqueous agar gels. This means that the agar-based gelled emulsions according to the invention will not fracture in the mouth and thus provide a more attractive chewing experience.

### Example 23—Syneresis Tests

Experiments were carried out to compare the syneresis of the agar-based emulsions according to Examples 20 and 21B (containing 1.5 wt. % and 2.0 wt. % agar, respectively) and that of a pure aqueous agar gel. The pure aqueous agar gel was made according to the same method as in Example 22. Each gel was subjected to a freeze-thaw cycle and the average weight loss was measured as described in the syneresis tests described herein. Results are shown in Table 8.

TABLE 8

| | Average weight loss | Standard |
|---|---|---|
| | (%) | deviation |
| 2.0% pure agar gel (3 samples) | 51.0 | 5.7 |
| Example 20 | 2.48 | 0.45 |
| Agar content: 1.5 wt. % (6 samples) | | |
| Example 21B | 2.24 | 0.13 |
| Agar content: 2.0 wt. % (3 samples) | | |

The pure agar gel lost over 50% of its original water content following the freeze-thaw cycle which indicates significant syneresis. The water loss for the gelled oil-in-water emulsions according to the invention is approx. 20-fold lower. The content of the agar did not significantly influence the extent of syneresis and both gelled emulsions according to the invention showed acceptable syneretic properties

### Example 24—Effect of Glycerol

The aqueous solvent for use in producing the formulations according to the invention may be modified by the incorporation of glycerol. Aqueous agar gels were produced in which water was successively exchanged with glycerol in order to determine the relative changes in the properties of the aqueous gel. The glycerol concentration was varied from 0 to 90 wt. % and water activity was measured as described herein. The results are shown in FIG. 6. At a 50:50 mixture of water:glycerol a water activity of below 0.8 was achieved. At this water activity, microbial growth is prevented. By using glycerol instead of water there could be a reduced need for sugar alcohols to reduce water activity and to obtain a product which is stable to microbial degradation.

G′ max and gelling temperature were also measured for the different gels according to the small scale deformation method described herein. With an increasing content of glycerol an increase in dynamic storage modulus was observed up to 50% inclusion of glycerol. The gelling temperature started to drop markedly around the same glycerol concentration. Large scale deformation and penetration showed an increase in resistance with increasing glycerol contents up to 50%. This is more or less in line with the small scale deformation results (G′). At the same time an increase of up to around 30% in compression distance before break was recorded at low and medium glycerol contents. This was also confirmed in a chewing test where the glycerol containing gels were perceived as being more “gelatin-like” than without.

### Example 25—Testing of Gelled oil-in-water Compositions with Higher Oil Content and Comparison with Gelatin-based oil-in-water Compositions

A gelled oil-in-water composition containing agar and faba bean protein and 40 wt. % oil was prepared according to Example 21B, but without paprika, lemon or stevia. The oil was added gradually, first up to 25 wt. %, then up to 30 wt. %, then 35 wt. %. In each step, the oil was first mixed in with a magnet or spatula before homogensiation using an ultra turrax machine. The final 5 wt. % oil (up to 40 wt. %) was incorporated by pure shaking due to very high viscosity at which the ultra turrax could not mix properly. A pure agar gel (without oil) was made as a comparison. An aqueous gelatin gel was also made for comparison using 260 Bloom type B bovine gelatin, 6.67 wt% in water. This was gelled at 4° C. overnight in cylinders and fully equilibrated to room temperature before texture measurements.

Large scale deformation measurements were carried out as described herein. The results for the agar-based compositions (without oil and with 40 wt. % oil) are provided in FIG. 7 and those for the gelatin gels are shown in FIG. 8. These are also provided in Table 9

TABLE 9

| | | Agar gelled | |
|---|---|---|---|
| | Agar gel - | emulsion - | |
| | no oil | 40 wt. % oil | Gelatin gel |
| Force at break (g) | 1734 ± 25 | 734 ± 21 | 1460 ± 139 |
| Strain at break | 29.3 ± 0.3 | 30 ± 1 | 73 ± 1 |
| Gradient (g/mm) | 196 ± 3 | 80 ± 2 | 9.4 ± 0 |
| Young's modulus (kPa) | 215 ± 4 | 88 ± 2 | 10.3 ± 0.3 |

The Young's modulus becomes more comparable to that of the gelatin gels in the agar gels containing 40 wt. % oil compared to without oil. The overall maximum gel strength decreases with the presence of oil, but shows the same large scale preservation of structure (relatively more so than without the oil present, although the sugar alcohols alone contribute quite significantly to the preserved structure at high strains).

Gelatin gels typically fail at much higher strain and show high resistance at high deformation, which compares to the deformation of chewing. It is the particular conservation of structure at strains above e.g. 40% that makes the agar gel formulations with oil and macromolecular surfactants according to the invention more comparable to gelatin.

Droplet size measurements were are also carried out in respect of the agar-based emulsions having different oil contents. The emulsions were slightly flocculated after dilution in water and SDS was added to deflocculate before droplet size measurements. Before measurement, successful deflocculation was confirmed with optical microscopy. Droplet sizes of the emulsions are given in Table 10.

TABLE 10

| | | | Dx | Dx | Dx |
|---|---|---|---|---|---|
| | D [4;3] | D [3;2] | (10) | (50) | (90) |
| faba 30 wt. % oil initial | 13.5 | 5.16 | 2.99 | 11.5 | 25.2 |
| faba 35 wt. % oil initial | 11.8 | 4.5 | 2.36 | 9.07 | 18.7 |
| faba 40 wt. % oil initial | 8.92 | 4.23 | 2.71 | 7.57 | 15.1 |
| faba 40 wt. % oil after | 9.78 | 4.46 | 3.03 | 7.95 | 17.2 |
| 3 hours at 55° C. | | | | | |
| faba 40 wt. % oil after | 16.8 | 6.43 | 4.25 | 13.6 | 33.8 |
| 20 hours at 55° C. | | | | | |

1. An orally administrable, gelled oil-in-water emulsion which is a self-supporting, viscoelastic solid having a gelled aqueous phase comprising a gelling agent which is agar, and wherein said emulsion is stabilised by a surfactant which is a plant-based protein, plant-based polysaccharide or derivative thereof.

2. An orally administrable, gelled oil-in-water emulsion as claimed in claim 1, wherein said surfactant is a plant-based protein or derivative thereof.

3. An orally administrable, gelled oil-in-water emulsion as claimed in claim 2, wherein said protein is obtained from a plant in the legume family, preferably from a pea or bean.

4. An orally administrable, gelled oil-in-water emulsion as claimed in claim 3, wherein said protein is faba bean protein or soy bean protein.

5. An orally administrable, gelled oil-in-water emulsion as claimed in claim 1, wherein said surfactant is a plant-based polysaccharide or derivative thereof.

6. An orally administrable, gelled oil-in-water emulsion as claimed in claim 5, wherein said surfactant is a hydrophobically-modified polysaccharide.

7. An orally administrable, gelled oil-in-water emulsion as claimed in claim 5 or claim 6, wherein said surfactant is a cellulose or a cellulose derivative, a starch or a starch derivative, or propylene glycol alginate.

8. An orally administrable, gelled oil-in-water emulsion as claimed in any one of the preceding claims which contains agar at concentration from about 0.1 to about 5 wt. % based on the total weight of the emulsion.

9. An orally administrable, gelled oil-in-water emulsion as claimed in any one of the preceding claims which further contains glycerol.

10. An orally administrable, gelled oil-in-water emulsion as claimed in any one of the preceding claims, wherein said aqueous phase constitutes from 50 to 95 wt. %, preferably from 55 to 90 wt. %, for example from 60 to 85 wt. %, from 70 to 85 wt. %, or from 75 to 80 wt. % of the emulsion.

11. An orally administrable, gelled oil-in-water emulsion as claimed in any one of the preceding claims, wherein the aqueous phase further comprises one or more bulking agents, for example sugar alcohols or sugars.

12. An orally administrable, gelled oil-in-water emulsion as claimed in claim 11, wherein said bulking agents are present at a concentration of from 45 to 70 wt. %, preferably 50 to 65 wt. %, e.g. 55 to 60 wt. %, based on the aqueous phase.

13. An orally administrable, gelled oil-in-water emulsion as claimed in any one of the preceding claims having an oil phase which comprises one or more physiologically tolerable lipids derived from rapeseed oil, sunflower oil, corn oil, olive oil, sesame oil, palm kernel oil, coconut oil, a nut oil, algae oil or hemp oil.

14. An orally administrable, gelled oil-in-water emulsion as claimed in any one of the preceding claims having an oil phase which constitutes from 5 to 50 wt. %, preferably from 10 to 45 wt. %, for example from 15 to 40 wt. %, from 15 to 30 wt. % or from 20 to 25 wt. %. of the emulsion.

15. An orally administrable, gelled oil-in-water emulsion as claimed in any one of the preceding claims which further comprises at least one pharmaceutically active agent.

16. An orally administrable, gelled oil-in-water emulsion as claimed in any one of the preceding claims which further comprises at least one nutraceutically active agent.

17. An orally administrable, gelled oil-in-water emulsion as claimed in claim 16, wherein said nutraceutically active agent is a vitamin or a mineral.

18. An orally administrable, gelled oil-in-water emulsion as claimed in any one of the preceding claims which is provided in unit dose form.

19. An orally administrable, gelled oil-in-water emulsion as claimed in claim 18, wherein said unit dose form is uncoated.

20. A package comprising an air-tight and light-tight compartment containing one dose unit of the gelled oil-in-water emulsion as claimed in claim 18 or claim 19.

21. A package as claimed in claim 20 which is a blister pack formed from a material which is not coated with a release agent.

22. A method for the preparation of an orally administrable, gelled oil-in-water emulsion as claimed in any one of claims 1 to 19, said method comprising the steps of: forming an oil phase which comprises one or more physiologically tolerable lipids; forming an aqueous phase comprising a gelling agent which is agar;
combining said oil phase and said aqueous phase to form an oil-in-water emulsion in the presence of a surfactant which is a plant-based protein, plant-based polysaccharide or derivative thereof; and allowing said emulsion to gel.

23. A gelled oil-in-water emulsion as claimed in any one of claims 1 to 19 for oral use as a medicament or for oral use in therapy.

24. A gelled oil-in-water emulsion as claimed in any one of claims 1 to 19 which contains at least one pharmaceutically active component for oral use in the treatment of a condition responsive to said pharmaceutically active component.

25. Use of a gelled oil-in-water emulsion as claimed in any one of claims 1 to 19 as a nutraceutical.
